SEQUENCE LISTING (10 sequences)

SEQ ID NO: 1 - protein, 111 aa, Homo sapiens
DIVLTQSPDSLAVSLGERATISCRASQSVSTSRYSYIHWYQQKPGQPPKLLIKYASNLESGVPSRFSGSGSGTDFTLNIHPLEPEDFATYYCHHSWEIPLTFGQGTKLEIK

SEQ ID NO: 2 - protein, 15 aa, Homo sapiens
RASQSVSTSRYSYIH

SEQ ID NO: 3 - protein, 7 aa, Homo sapiens
YASNLES

SEQ ID NO: 4 - protein, 9 aa, Homo sapiens
HHSWEIPLT

SEQ ID NO: 5 - protein, 124 aa, Homo sapiens
EVKVVESGGGLVQPGGSMKLSCVVSGFTFSNYWVNWVRQAPGKGLEWVAQIRLKSDNYATHYEESVKGRFTISRDDSKSSVYLQMNNLRAEDSGIYYCTNWEDYWGQGTTVTVSSASTKGPSVF

SEQ ID NO: 6 - protein, 5 aa, Homo sapiens
NYWVN

SEQ ID NO: 7 - protein, 19 aa, Homo sapiens
QIRLKSDNYATHYEESVKG

SEQ ID NO: 8 - protein, 4 aa, Homo sapiens
WEDY

SEQ ID NO: 9 - protein, 218 aa, Homo sapiens
DIVLTQSPDSLAVSLGERATISCRASQSVSTSRYSYIHWYQQKPGQPPKLLIKYASNLESGVPSRFSGSGSGTDFTLNIHPLEPEDFATYYCHHSWEIPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 442 aa, Homo sapiens
EVKVVESGGGLVQPGGSMKLSCVVSGFTFSNYWVNWVRQAPGKGLEWVAQIRLKSDNYATHYEESVKGRFTISRDDSKSSVYLQMNNLRAEDSGIYYCTNWEDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2021/0023216 A1
(43) Pub. Date: Jan. 28, 2021

(54) STABLE AQUEOUS ANTI-TAU ANTIBODY FORMULATIONS

(71) Applicant: ABBVIE DEUTSCHLAND GMBH & CO. KG, Wiesbaden (DE)

(72) Inventors: Jonas ANGSTENBERGER, Ludwigshafen (DE); Kerstin APPENZELLER, Ludwigshafen (DE); Martin HULSMEYER, Ludwigshafen (DE); Katharina KALETA, Ludwigshafen (DE); Tanja MEYER, Ludwigshafen (DE); Christian RIED, Ludwigshafen (DE); Christine RINN, Ludwigshafen (DE); Kathrin SCHAKER-THEOBALD, Ludwigshafen (DE); Michael SIEDLER, Ludwigshafen (DE); Axel WILBERTZ, Ludwigshafen (DE)

(21) Appl. No.: 17/040,496

(22) PCT Filed: Mar. 22, 2019
(86) PCT No.: PCT/EP2019/057343

§ 371 Date: Sep. 22, 2020

Related U.S. Application Data
(60) Provisional application No. 62/647,615, filed on Mar. 23, 2018.

(51) Int. Cl. A61K 39/395; C07K 16/18; A61K 47/22; A61K 47/26

(52) U.S. Cl. CPC A61K 39/39591; A61K 47/26; A61K 47/22; C07K 16/18

(57) ABSTRACT

The present disclosure provides stable, aqueous, buffered compositions comprising a high concentration of ABBV-8E12, such as a stable, aqueous buffered composition including ABBV-8E12 at a concentration of about 100 mg/ml, at least one buffer, an excipient, a surfactant, and optionally an antioxidant.

### CROSS-REFERENCE TO RELATED APPLICATION

This application claims the benefit of U.S. Provisional Application No. 62/647,615, filed Mar. 23, 2018, the entire disclosure of which is incorporated by reference herein in its entirety.

### FIELD

The present disclosure relates generally to stable aqueous anti-tau antibody formulations, including stable aqueous formulations of the ABBV-8E12 anti-tau antibody having a high protein concentration.

### BACKGROUND

Tauopathies have in common the accumulation of insoluble, hyper-phosphorylated tau protein in the brain. More than twenty different neurodegenerative disorders are characterized by some degree of neurofibrillary degeneration and can be classified as tauopathies (Williams 2006). Prototypical tauopathies, such as progressive supranuclear palsy (PSP) and corticobasal degeneration (CBD) are characterized by tau inclusions being the sole or predominant central nervous system lesions. Prototypical tauopathies differ from other tauopathies where tau aggregates are found in the presence of other neuropathological features, like the amyloid beta (Aβ) plaques found in Alzheimer's Disease (AD) or the Lewy bodies found in Parkinson's Disease (PD). In these non-prototypical tauopathies, it is more uncertain if the tau pathology represents the primary disease driver or if it is secondary to other protein misfolding and neurodegeneration.

Strong experimental evidence and biological rationale exists to support the tau immunotherapy strategy as a way to counter tau pathology in neurodegeneration. First, tau is normally a highly soluble, natively unfolded, and intracellular protein, so an extracellular antibody is unlikely to affect the normal functions of tau. Second, the burden of tau pathology correlates with progressive neuronal dysfunction, synaptic loss, and functional decline in humans and transgenic mouse models of tauopathy. Third, under pathological conditions, tau becomes misfolded and aggregates into intraneuronal neurofibrillary tangles (NFTs) composed of pathological tau fibrils. In human tauopathies, this pathology progresses from one brain region to another in disease-specific patterns. Experimental data suggests that tau aggregates can spread from cell to cell to induce further tau aggregation and spreading of tau pathology in brain. This data suggests that aggregates produced in one cell are released into the extracellular space and can promote aggregation in neighboring or connected cells. Finally, it has been demonstrated that anti-tau antibodies can prevent or slow the progression of tau pathology in the brain of mice that carry mutated human form of tau. Anti-tau antibodies have been described, for example, in U.S. Patent Application Publication No. 2017/0058024 and U.S. Patent No. 2015/0183855, the entire disclosures of which are incorporated by reference herein in their entireties. ABBV-8E12, also known as C2N-8E12, is a humanized monoclonal anti-tau antibody.

Often protein-based pharmaceutical products need to be formulated at high concentrations for therapeutic efficacy. Highly concentrated protein formulations are desirable for therapeutic uses since they allow for dosages with smaller volumes, limiting patient discomfort, and are more economically packaged and stored. The development of high protein concentration formulations, however, presents many challenges, including manufacturing, stability, analytical, and, especially for therapeutic proteins, delivery challenges. For example, difficulties with the aggregation, insolubility and degradation of proteins generally increase as protein concentrations in formulations are raised (for review, see Shire, S. J. et al. J. Pharm. Sci., 93, 1390 (2004)). Previously unseen negative effects may be caused by additives that, at lower concentrations of the additives or the protein, provided beneficial effects. The production of high concentration protein formulations may lead to significant problems with opalescence, aggregation and precipitation. In addition to the potential for normative protein aggregation and particulate formation, reversible self-association may occur, which may result in increased viscosity or other properties that complicate delivery by injection. High viscosity also may complicate manufacturing of high protein concentrations by filtration approaches.

Thus, pharmaceutical protein formulations typically carefully balance ingredients and concentrations to enhance protein stability and therapeutic requirements while limiting any negative side-effects.

As proteins and other biomacromolecules gain increased interest as drug molecules, formulations for delivering such molecules are becoming an important issue. Despite the revolutionary progress in the large-scale manufacturing of proteins for therapeutic use, effective and convenient delivery of these agents in the body remains a major challenge due to their intrinsic physicochemical and biological properties, including poor permeation through biological membranes, large molecular size, short plasma half life, self association, physical and chemical instability, aggregation, adsorption, and immunogenicity.

Accordingly, there remains a need for stable aqueous anti-tau antibody formulations at a high concentration, including stable aqueous ABBV-8E12 formulations at a high concentration.

### SUMMARY

In embodiments, the present disclosure provides a stable aqueous, buffered composition comprising an antibody at a concentration of about 100 mg/ml. In embodiments, the antibody comprises a variable heavy chain that comprises a CDR1 sequence of SEQ ID NO: 6, a CDR2 sequence of SEQ ID NO:7, and a CDR3 sequence of SEQ ID NO: 8; and a variable light chain that comprises a CDR1 sequence of SEQ ID NO:2, a CDR2 sequence of SEQ ID NO: 3, and a CDR3 sequence of SEQ ID NO: 4. In embodiments, the antibody comprises a light chain comprising the amino acid sequence set forth in SEQ ID NO: 9 and a heavy chain comprising the amino acid sequence set forth in SEQ ID NO: 10. In embodiments, the antibody is ABBV-8E12. In embodiments, the composition further comprises at least one buffer, an excipient, a surfactant, and optionally an anti-oxidant. In embodiments, the buffer is selected from the group consisting of histidine, phosphate, and succinate buffer. In embodiments, the buffer is histidine. In embodiments, the surfactant is a polysorbate, such as polysorbate 20 or polysorbate 80. In embodiments, the surfactant is polysorbate 80. In embodiments, the excipient may be a salt (such as NaCl), a polyol (such as mannitol, sorbitol, or trehalose), or a sugar (such as sucrose, glucose, or dextrose). In embodiments, the pH is about 5 to about 7. In embodiments, the viscosity of the composition is less than about 20 mPas at room temperature, such as less than 10 mPas, or less than 5 mPas.

In embodiments, the present disclosure provides a stable, aqueous buffered composition containing ABBV-8E12 at a concentration of about 100 mg/ml. In embodiments, the composition further contains at least one buffer and a surfactant. The composition may also contain at least one excipient and/or an anti-oxidant. In embodiments, the polysorbate is polysorbate 20 or polysorbate 80. In embodiments, the excipient may be a salt (such as NaCl), a polyol (such as sorbitol), or a sugar (such as sucrose). In embodiments, the anti-oxidant may be an amino acid, such as methionine. In embodiments, the buffer is selected from the group consisting of histidine, phosphate, and succinate. In embodiments, the buffer is histidine.

In embodiments, the present disclosure provides a stable aqueous buffered composition comprising ABBV-8E12 at a concentration of about 100 mg/ml, histidine, a polysorbate, an excipient, and optionally an anti-oxidant. In embodiments, the polysorbate is polysorbate 20 or polysorbate 80. In embodiments, the polysorbate is polysorbate 80.

In embodiments, the present disclosure provides a stable, aqueous, buffered composition containing ABBV-8E12 at a concentration of about 100 mg/ml, histidine, sucrose, and a polysorbate. In embodiments, the polysorbate is polysorbate 20 or polysorbate 80. In embodiments, the polysorbate is polysorbate 80.

### BRIEF DESCRIPTION OF THE DRAWINGS

FIG. 1 shows the variable region sequences of the murine HJ8.5 anti-tau antibody, as well as 4 humanized variant sequences for each of the heavy and light chains (4 VH and 4 VL/VK sequences). CDR sequences are underlined, and framework changes from the original mouse sequences are in bold.

FIG. 2 shows the sequences of the humanized variable and constant region sequences for each of the heavy and light chains (4 VH and 4 VL/VK sequences). The variable heavy chain is grafted to the constant heavy chain of human IgG4 containing a S241P hinge stabilizing mutation.

FIG. 3 shows the turbidity of ABBV-8E12 samples formulated at 100 mg/ml following freeze/thaw stress (FIG. 3A) and following freeze/thaw and mechanical stress (FIG. 3B). Application of mechanical stress leads to higher turbidity, especially for the samples formulated without polysorbate 20 or polysorbate 80.

FIG. 4 shows the turbidity of 100 mg/ml ABBV-8E12 samples formulated with polysorbate following temperature storage at 5° C., 25° C., and 40° C. Samples formulated in histidine and phosphate buffer showed the lowest turbidities, as did samples formulated with NaCl.

FIG. 5 shows a main effects plot of turbidity after application of freeze/thaw and mechanical stress. Turbidity increases through the application of mechanical stress, especially for samples without surfactant.

FIG. 6 shows monomer contents of all ABBV-8E12 samples, as measured by SE-UHPLC. Samples formulated in histidine buffer show the highest monomer content.

FIG. 7 shows high molecular weight aggregates as measured by SE-UHPLC.

FIG. 8 shows measured reversible aggregation, as measured by UHPLC.

FIG. 9A-C show SEC % monomer data for various ABBV-8E12 formulations for long-term stability analysis, as shown by SEC main peak data at 4° C. (FIG. 9A), 25° C. (FIG. 9B), and 40° C. (FIG. 9C).

FIG. 10A-C show SEC % aggregates (high molecular weight) for various ABBV-8E12 formulations for long-term stability analysis, as shown by SEC HMW Peak data at 4° C. (FIG. 10A), 25° C. (FIG. 10B), and 40° C. (FIG. 10C).

FIG. 11A-C show CE reduced % purity data for 4° C. incubated samples of ABBV-8E12 formulations (FIG. 15A), 25° C. incubated samples (FIG. 15B) and 40° C. incubated samples (FIG. 15C) for long-term stability analysis.

FIG. 12A-C show CE non-reduced % main data for 4° C. incubated samples of ABBV-8E12 formulations (FIG. 16A), 25° C. incubated samples (FIG. 16B), and 40° C. incubated samples (FIG. 16C) for long-term stability analysis.

### DETAILED DESCRIPTION

The preparation of high concentration protein formulations can lead to problems with opalescence and stability. When aqueous pharmaceutical compositions comprising a high protein concentration (e.g., 100 mg/ml) of ABBV-8E12 are stored on a long-term basis, the activity of ABBV-8E12 can be lost or decreased due to aggregation and/or degradation. The present disclosure provides for aqueous formulations of ABBV-8E12 at a high protein concentration (e.g., 100 mg/ml) that allow for long-term storage of ABBV-8E12, so that ABBV-8E12 is stable over the course of storage, either in liquid or frozen states. As discussed below and shown in the examples of the present disclosure, the aqueous formulations of the present disclosure overcome issues of stability and turbidity to provide stable, high-concentration formulations of ABBV-8E12. The provided formulations do not require any additional steps, such as rehydrating.

Various embodiments of the disclosure are now described in detail. As used in the description and throughout the claims, the meaning of “a”, “an”, and “the” includes plural reference unless the context clearly dictates otherwise. Also, as used in the description and throughout the claims, the meaning of “in” includes “in” and “on” unless the context clearly dictates otherwise. Additionally, some terms used in this disclosure are more specifically defined below.

The term “aqueous formulation” refers to a solution in which the solvent is water.

The term “antibody”, as used herein, refers to immunoglobulin molecules comprised of four polypeptide chains, two heavy (H) chains and two light (L) chains inter-connected by disulfide bonds. Each heavy chain is comprised of a heavy chain variable region (“HCVR” or “VH”) and a heavy chain constant region. The heavy chain constant region is comprised of three domains (CH1, CH2, and CH3). Each light chain is comprised of a light chain variable region (“LCVR” or “VL”) and a light chain constant region. The light chain constant region is comprised of one domain (CL). The VH and VL regions can be further subdivided into regions of hypervariability, terms complementarity determining regions (CDR) interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In embodiments, the formulation contains an antibody with amino acid sequences as set forth in WO2016/201434, the entire disclosure of which is hereby incorporated by reference herein in its entirety.

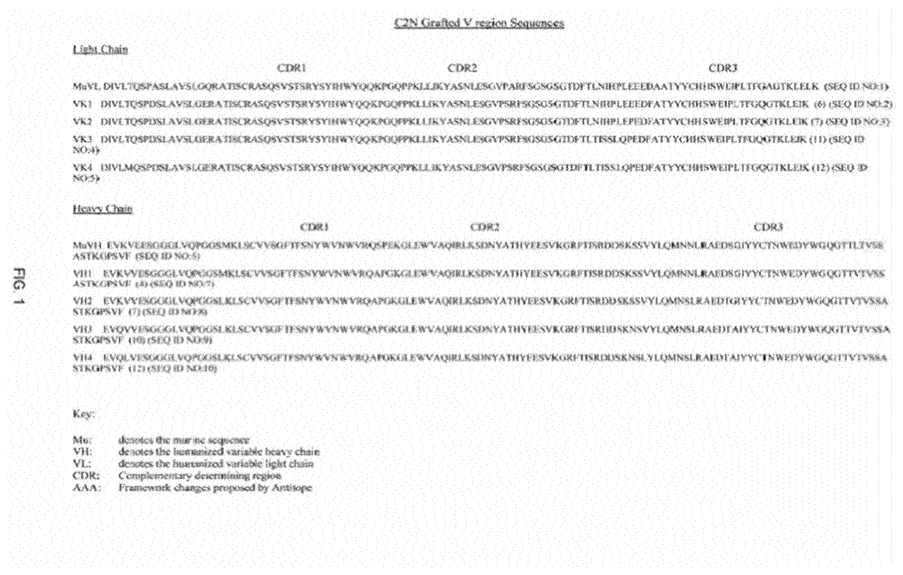

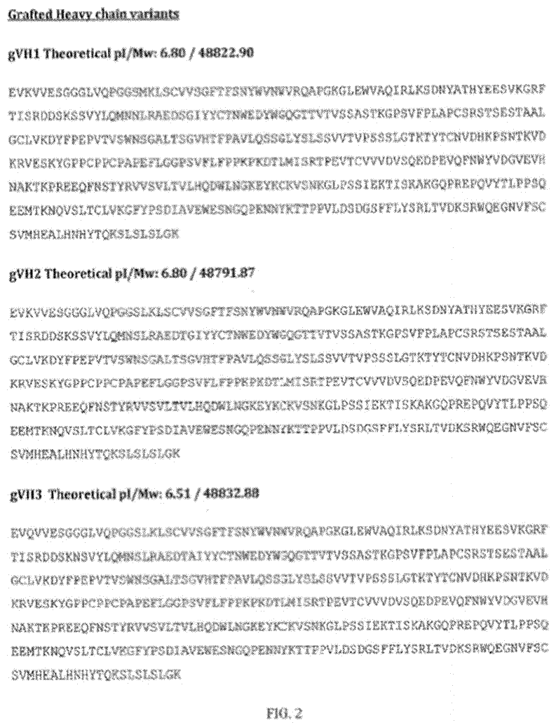

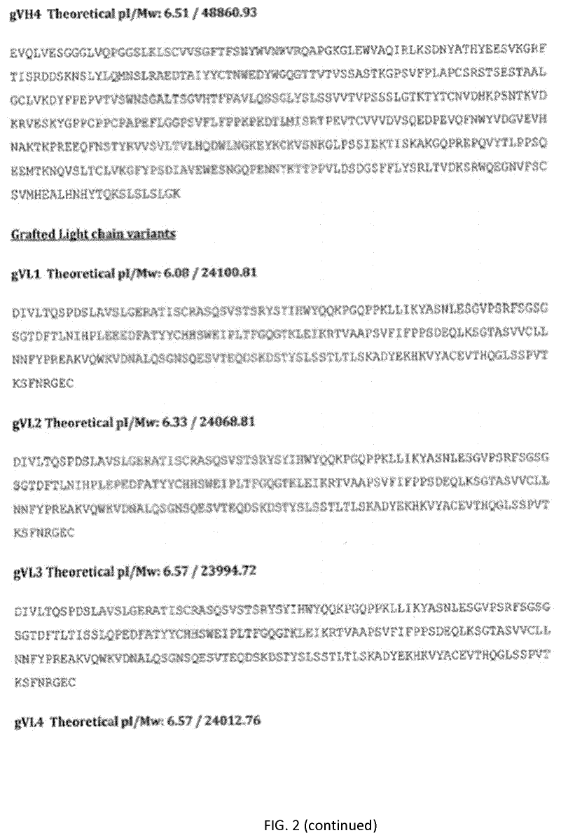

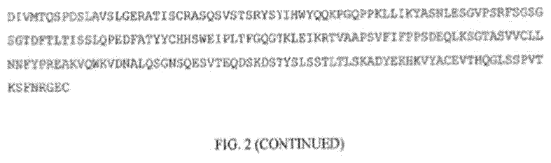

The term “ABBV-8E12” is synonymous with the term “C2N-8E12”, and refers to a humanized recombinant IgG4 anti-human tau antibody comprising a heavy chain (VH) region and a light chain (VL) region, having amino acid sequences as set forth in FIG. 1 and FIG. 2. ABBV-8E12 comprises a LCDR1, LCDR2, and LCDR3 as set forth in SEQ ID NO: 2, 3, and 4 respectively, and a HCDR1, HCDR2 and HCDR3 as set forth in SEQ ID NO: 6, 7, and 8. ABBV-8E12 comprises a light chain variable region as set forth in SEQ ID NO: 1 and a heavy chain variable region as set forth in SEQ ID NO: 5. ABBV-8E12 comprises a light chain comprising the amino acid sequence set forth in SEQ ID NO: 9 and a heavy chain comprising the amino acid sequence set forth in SEQ ID NO: 10.

For purposes of the present disclosure, “ABBV-8E12” also encompasses ABBV-8E12 with minor modifications in the amino acid structure (including additions, deletions, and/or substitutions of amino acids) or in the glycosylation properties that do not significantly affect function of the polypeptide.

The term “isolated antibody” refers to an antibody that is substantially free of other antibodies having different antigenic specificities (e.g., an isolated antibody that specifically binds tau is substantially free of antibodies that specifically bind antigens other than tau). Moreover, an isolated antibody may be substantially free of other cellular material and/or chemicals.

The term “glycine” refers to an amino acid whose codons are GGT, GGC, GGA, and GGG.

The term “arginine” refers to an α-amino acid whose codons are CCU, CCC, CCA, and CCG.

The term “alanine” refers to an amino acid whose codons are GCT, GCC, GCA, and GCG.

The term “methionine” refers to an amino acid whose codon is ATG.

The term “Glutamate” refers to an amino acid whose codons are GAA and GAG.

The term “sugar” refers to monosaccharides, disaccharides, and polysaccharides. Examples of sugars include, but are not limited to, sucrose, glucose, dextrose, and others.

The term “polyol” refers to an alcohol containing multiple hydroxyl groups. Examples of polyols include, but are not limited to, mannitol, sorbitol, and others.

The term “metal ion” refers to a metal atom with a net positive or negative electrical charge. For purposes of the present disclosure, the term “metal ion” also includes sources of metal ions, including but not limited to metal salts.

The term “long-term storage” or “long term stability” is understood to mean that the pharmaceutical composition can be stored for at least three months (i.e., three months or more), such as more than six months (six months or more), or more than a year, or more than two years. Long-term storage is also understood to mean that the pharmaceutical composition is stored as a liquid or is frozen. It is also contemplated that the composition can be frozen and thawed more than once.

The term “stable” with respect to long-term storage is understood to mean that ABBV-8E12 within the pharmaceutical composition does not lose more than 20%, or more preferably 15%, or 10%, or 5% of its activity relative to the activity of the composition at the beginning of storage.

The term “substantially free” means that either the no substance is present, or only minimal, trace amounts of the substance are present which do not have any substantial impact on the properties of the composition. If reference is made to no amount of a substance, it should be understood as “no detectable amount”.

The term “mammal” includes, but is not limited to, a human.

The term “pharmaceutically acceptable carrier” refers to a non-toxic solid, semisolid, or liquid filler, diluent, encapsulating material, formulation auxiliary, or excipient of any conventional type. A pharmaceutically acceptable carrier is non-toxic to recipients at the dosages and concentrations employed and is compatible with other ingredients of the formulation.

The term “composition” refers to a mixture that usually contains a carrier, such as a pharmaceutically acceptable carrier or excipient that is conventional in the art and which is suitable for administration to a subject for therapeutic, diagnostic, or prophylactic purposes. It may include a cell culture in which the polypeptide or polynucleotide is present in the cells or the culture medium. For example, compositions for oral administration can form solutions, suspensions, tablets, pills, capsules, sustained release formulations, oral rinses, or powders.

The terms “pharmaceutical composition” and “formulation” are used interchangeably.

The term “treatment” refers to any administration or application of remedies for disease in a mammal and includes inhibiting the disease, arresting its development, relieving the disease, for example, by causing regression, or restoring or repairing a lost, missing, or defective function; or stimulating an inefficient process. The term includes obtaining a desired pharmacologic and/or physiologic effect, covering any treatment of a pathological condition or disorder in a mammal. The effect may be prophylactic in terms of completely or partially preventing a disorder or symptom thereof and/or may be therapeutic in terms of a partial or complete cure for a disorder and/or adverse effect attributable to the disorder. It includes (1) preventing the disorder from occurring or recurring in a subject who may be predisposed to the disorder but is not yet symptomatic, (2) inhibiting the disorder, such as arresting its development, (3) stopping or terminating the disorder or at least its associated symptoms, so that the host no longer suffers from the disorder or its symptoms, such as causing regression of the disorder or its symptoms, for example, by restoring or repairing a lost, missing or defective function, or stimulating an inefficient process, or (4) relieving, alleviating or ameliorating the disorder, or symptoms associated therewith, where ameliorating is used in a broad sense to refer to at least a reduction in the magnitude of a parameter, such as inflammation, pain and/or tumor size.

The term “disease” refers to any condition, infection, disorder or syndrome that requires medical intervention or for which medical intervention is desirable. Such medical intervention can include treatment, diagnosis and/or prevention.

The term “therapeutically effective amount” refers to an amount which, when administered to a living subject, achieves a desired effect on the living subject. For example, an effective amount of the antibody of the present disclosure for administration to the living subject is an amount that prevents and/or treats a tauopathy, such as Alzheimer's Disease or Progressive Supranuclear Palsy. The exact amount will depend on the purpose of the treatment, and will be ascertainable by one skilled in the art using known techniques. As is known in the art, adjustments for systemic versus localized delivery, age, body weight, general health, sex, diet, time of administration, drug interaction and the seventy of the condition may be necessary, and will be ascertainable with routine experimentation by those skilled in the art.

The term “tauopathy” refers to a neurodegenerative disease characterized by accumulation of hyperphosphorylated tau protein in the brain. Tauopathies include, for example, Alzheimer's Disease or Progressive Supranuclear Palsy.

### Embodiments

Embodiments of the present disclosure are explained in more detail below.

The compositions of the present disclosure comprise ABBV-8E12. As discussed above, ABBV-8E12 (also known as C2N-8E12) is a humanized recombinant IgG4 antibody that specifically binds human tau. ABBV-8E12 has been described, for example, in WO2016/201434, the entire disclosure of which is incorporated by reference herein in its entirety. ABBV-8E12 suitable for storage in the present disclosure can be prepared, for example, by suitable methods known in the art. Purification of ABBV-8E12 can be performed by any suitable standard method. When ABBV-8E12 is produced intracellularly, the particulate debris may be removed, for example, by centrifugation or ultrafiltration. When ABBV-8E12 is secreted into the medium, supernatants from such expression systems can be first concentrated using standard polypeptide concentration filters. Protease inhibitors can also be added to inhibit proteolysis and antibiotics can be included to prevent the growth of microorganisms.

ABBV-8E12 can be purified using, for example, hydroxyapatite chromatography, gel electrophoresis, dialysis, affinity chromatography, and any combination of other suitable purification techniques, including but not limited to Protein A chromatography, fractionation on an ion-exchange column, ethanol precipitation, reverse phase HPLC, chromatography on silica, chromatography on heparin SEPHAROSET®, an anion or cation exchange resin chromatography (such as a polyaspartic acid column), chromatofocusing, CDS-PAGE, and ammonium sulfate precipitation.

In embodiments, the present disclosure provides a stable aqueous pharmaceutical composition comprising: an antibody comprising a variable heavy chain that comprises a CDR1 sequence of SEQ ID NO: 6, a CDR2 sequence of SEQ ID NO:7, and a CDR3 sequence of SEQ ID NO: 8; and a variable light chain that comprises a CDR1 sequence of SEQ ID NO:2, a CDR2 sequence of SEQ ID NO: 3, and a CDR3 sequence of SEQ ID NO: 4; a surfactant; a buffer; optionally an excipient; and optionally an antioxidant, wherein the concentration of the antibody is about 100 mg/ml. In embodiments, the antibody comprises a light chain variable region as set forth in SEQ ID NO: 1 and a heavy chain variable region as set forth in SEQ ID NO: 5. In embodiments, the antibody comprises a light chain comprising the amino acid sequence set forth in SEQ ID NO: 9 and a heavy chain comprising the amino acid sequence set forth in SEQ ID NO: 10.

In embodiments, the present disclosure provides a stable aqueous pharmaceutical composition comprising ABBV-8E12, a surfactant, a buffer, optionally an excipient and optionally an antioxidant, in which the concentration of ABBV-8E12 is about 100 mg/mL. In embodiments of the present disclosure comprising a buffer, the buffer may be selected from acetate, citrate, gluconate, phosphate, succinate, histidine, tartrate, maleate, adipic acid, lactic acid, or trishydroxymethylaminomethane (tris). In embodiments, the buffer is histidine, phosphate, or succinate. In embodiments, the buffer is histidine.

In embodiments, the buffer is present at a concentration from about 5 mM to about 50 mM, such as from about 10 to about 40 mM, or from about 20 to about 30 mM, or about 25 mM.

In embodiments, the pH of the compositions is from about 5 to about 7, such as from about 5.5 to about 6.5, or about 6.1.

The compositions of the present disclosure are stable at a high protein concentration (e.g., about 100 mg/mL) while maintaining a viscosity suitable for injection, such as injection through a needle or other suitable device at room temperature (e.g., about 20 to about 25° C.). In embodiments, the compositions maintain a viscosity suitable for injection through a needle or other suitable device in a range of 27 gauge to 31 gauge, such as 28 to 31 gauge, or 29 to 31 gauge, or 29 gauge in size while at room temperature (such as from about 20° C. to about 25° C.). In embodiments, the composition of the present disclosure have a viscosity of less than 20 mPas at room temperature (e.g., 20-25° C.) at ABBV-8E12 concentrations of about 100 mg/mL, such as from about 1 to about 20 mPas, or from about 1 to about 15 mPas, or from about 1 to about 10 mPas. In embodiments, the compositions of the present disclosure have a viscosity of less than 10 mPas at room temperature (e.g., 20-25° C.) for ABBV-8E12 concentrations of about 100 mg/mL, such as from about 1 to about 10 mPas, or from about 1 to about 8 mPas, or from about 1 to about 5 mPas. In embodiments, the compositions of the present disclosure have a viscosity of less than 5 mPas at room temperature (e.g., 20-25° C.) for ABBV-8E12 concentrations of about 100 mg/mL. In embodiments, the compositions of the present disclosure have a viscosity of about 4.5 mPas.

In embodiments, the present disclosure provides a stable aqueous pharmaceutical composition comprising: an antibody comprising a LCDR1, LCDR2, and LCDR3 as set forth in SEQ ID NO: 2, 3, and 4 respectively, and a HCDR1, HCDR2 and HCDR3 as set forth in SEQ ID NO: 6, 7, and 8; a surfactant; a buffer; optionally an excipient; and optionally an antioxidant, wherein the concentration of the antibody is about 100 mg/ml, and wherein the composition has a viscosity of less than about 20 mPas at room temperature. In embodiments, the antibody comprises a light chain variable region as set forth in SEQ ID NO: 1 and a heavy chain variable region as set forth in SEQ ID NO: 5. In embodiments, the antibody comprises a light chain comprising the amino acid sequence set forth in SEQ ID NO: 9 and a heavy chain comprising the amino acid sequence set forth in SEQ ID NO: 10. In embodiments, the antibody is ABBV-8E12. In embodiments, the viscosity is less than about 10 mPas at room temperature. In embodiments, the viscosity is less than about 5 mPas at room temperature.

In embodiments, the surfactant is present in an amount of from about 0.1 to about 10 mg/mL, such as from about 0.3 to about 5 mg/mL, or from about 0.5 to about 1.5 mg/mL, or about 1 mg/mL. In embodiments, the surfactant is a polysorbate surfactant. For example, in embodiments, the surfactant is polysorbate 20 or polysorbate 80. In embodiments, the surfactant is polysorbate 80.

In embodiments, the formulation composition comprises at least one additional excipient. In embodiments, the at least one additional excipient is a stabilizer. In embodiments, the at least one additional excipient is selected from the group consisting of a salt, a sugar, or a polyol.

In embodiments, the at least one additional excipient is a salt, such as sodium chloride. In embodiments, the salt is present in an amount of from about 3 to about 20 mg/ml, such as from about 5 to about 10 mg/ml, or from about 8 to about 10 mg/ml, or about 8 mg/ml.

In embodiments, the at least one additional excipient is a polyol. In embodiments, the polyol is a sugar alcohol, such as mannitol, sorbitol, or trehalose. In embodiments, the polyol is present in an amount of from about 30 to about 50 mg/ml, or from about 35 to about 45 mg/ml, or from about 38 to about 43 mg/ml, or about 42 mg/ml.

In embodiments, the at least one additional excipient is a sugar. In embodiments, the sugar is selected from the group consisting of sucrose, glucose, or dextrose. In embodiments, the sugar is present in an amount of from about 50 to about 100 mg/ml, such as from about 60 to about 90 mg/ml, or from about 70 to about 80 mg/ml, or about 75 mg/ml.

In embodiments, the formulation may contain an antioxidant. In embodiments, the antioxidant is an amino acid. In embodiments, the amino acid is selected from the group consisting of glycine, alanine, glutamate, arginine, and methionine. In embodiments, the amino acid is selected from the group consisting of glycine, arginine, and methionine. In embodiments, the amino acid is methionine. In embodiments, the antioxidant is present in an amount of from about 5 to about 15 mM, or from about 8 to about 12 mM, or about 10 mM.

In embodiments, the present disclosure provides a stable aqueous pharmaceutical composition comprising ABBV-8E12 at a concentration of about 100 mg/mL; a buffer selected from the group consisting of histidine, phosphate, and succinate; a surfactant; an excipient; and optionally an antioxidant. In embodiments, the buffer is histidine. In embodiments, the surfactant is a polysorbate, and in embodiments, the surfactant is polysorbate 80. In embodiments, the excipient is selected from the group consisting of a salt, a polyol, or a sugar.

In embodiments, the present disclosure provides a stable aqueous pharmaceutical composition comprising ABBV-8E12 at a concentration of about 100 mg/mL; a buffer selected from the group consisting of histidine, phosphate, and succinate; a surfactant, wherein the surfactant is a polysorbate; and an excipient selected from the group consisting of a salt, a polyol, or a sugar. In embodiments, the polysorbate is polysorbate 80.

In embodiments, the present disclosure provides a stable aqueous pharmaceutical composition comprising ABBV-8E12 at a concentration of about 100 mg/ml; histidine; a polysorbate; optionally an excipient, and optionally an antioxidant. In embodiments, the polysorbate is polysorbate 20 or polysorbate 80. In embodiments, the polysorbate is polysorbate 80. In embodiments, the excipient is selected from the group consisting of a salt, a polyol, or a sugar.

In embodiments, the present disclosure provides a stable aqueous pharmaceutical composition comprising ABBV-8E12 at a concentration of about 100 mg/mL; a buffer selected from the group consisting of histidine, phosphate, and succinate at a concentration of about 5 to 50 mM; a surfactant at a concentration of 1 to 10 mg/mL, wherein the surfactant is a polysorbate; and an excipient selected from the group consisting of a salt, a polyol, or a sugar. In embodiments, the polysorbate is selected from the group consisting of polysorbate 20 and polysorbate 80. In embodiments, the polysorbate is polysorbate 80. In embodiments, the buffer is histidine. In embodiments, the excipient is sucrose.

In embodiments, the present disclosure provides a stable aqueous pharmaceutical formulation comprising ABBV-8E12 at a concentration of about 100 mg/ml; a buffer selected from the group consisting of histidine, phosphate, and succinate; a surfactant, wherein the surfactant is a polysorbate; and an excipient selected from the group consisting of sodium chloride, sorbitol, and sucrose. In embodiments, the surfactant is polysorbate 20 or polysorbate 80. In embodiments the surfactant is polysorbate 80. In embodiments, the buffer is histidine. In embodiments, the excipient is sucrose.

In embodiments, the present disclosure provides a stable aqueous pharmaceutical formulation comprising ABBV-8E12 at a concentration of about 100 mg/ml; a buffer selected from the group consisting of histidine, phosphate, and succinate at a concentration of about 5 to 50 mM; a surfactant at a concentration of 1 to 10 mg/ml, wherein the surfactant is a polysorbate; and an excipient selected from the group consisting of sodium chloride, sorbitol, and sucrose. In embodiments, the surfactant is polysorbate 20 or polysorbate 80. In embodiments, the surfactant is polysorbate 80. In embodiments, the excipient is sucrose.

In embodiments, the present disclosure provides a stable aqueous pharmaceutical formulation comprising ABBV-8E12 at a concentration of about 100 mg/ml; a buffer selected from the group consisting of histidine, phosphate, and succinate; a surfactant, wherein the surfactant is a polysorbate; and a salt. In embodiments, the salt is sodium chloride. In embodiments, the buffer is histidine. In embodiments, the surfactant is polysorbate 20 or polysorbate 80. In embodiments, the surfactant is polysorbate 80. For example, in embodiments, the present disclosure provides a stable aqueous pharmaceutical formulation comprising ABBV-8E12 at a concentration of about 100 mg/ml; a buffer selected from the group consisting of histidine, phosphate, and succinate at a concentration of about 5 to 50 mM; a surfactant, wherein the surfactant is a polysorbate, at a concentration of 1 to 10 mg/ml; and a salt at a concentration of from about 3 to about 20 mg/ml.

In embodiments, the present disclosure provides a stable aqueous pharmaceutical formulation comprising ABBV-8E12 at a concentration of about 100 mg/ml; histidine; a polysorbate; and sodium chloride. In embodiments, the polysorbate is polysorbate 20 or polysorbate 80. In embodiments, the polysorbate is polysorbate 80.

In embodiments, the present disclosure provides a stable aqueous pharmaceutical formulation comprising ABBV-8E12 at a concentration of about 100 mg/ml; a buffer selected from the group consisting of histidine, phosphate, and succinate; a surfactant, wherein the surfactant is a polysorbate; and a polyol. In embodiments, the polyol is mannitol, sorbitol, or trehalose. In embodiments, the surfactant is polysorbate 20 or polysorbate 80. In embodiments, the surfactant is polysorbate 80. In embodiments, the present disclosure provides a stable aqueous pharmaceutical formulation comprising ABBV-8E12 at a concentration of about 100 mg/ml; a buffer selected from the group consisting of histidine, phosphate, and succinate at a concentration of about 5 to 50 mM; a surfactant, wherein the surfactant is a polysorbate, at a concentration of 1 to 10 mg/mL; and a polyol at a concentration of from about 30 to about 50 mg/ml.

For example, in embodiments, the present disclosure provides a stable aqueous pharmaceutical formulation comprising ABBV-8E12 at a concentration of about 100 mg/ml, histidine, a polysorbate, and sorbitol. In embodiments, the polysorbate is polysorbate 20 or polysorbate 80 In embodiments, the polysorbate is polysorbate 80.

In embodiments, the present disclosure provides a stable aqueous pharmaceutical formulation comprising ABBV-8E12 at a concentration of about 100 mg/ml; a buffer selected from the group consisting of histidine, phosphate, and succinate; a surfactant, wherein the surfactant is a polysorbate; and a sugar. In embodiments, the sugar is sucrose, glucose, or dextrose. In embodiments, the surfactant is polysorbate 20 or polysorbate 80. In embodiments, the surfactant is polysorbate 80. In embodiments, the buffer is histidine. For example, in embodiments, the present disclosure provides a stable aqueous pharmaceutical formulation comprising ABBV-8E12 at a concentration of about 100 mg/ml; a buffer selected from the group consisting of histidine, phosphate, and succinate at a concentration of about 5 to 50 mM; a surfactant, wherein the surfactant is a polysorbate, at a concentration of 1 to 10 mg/mL; and a sugar at a concentration of about 50 to about 100 mg/ml.

For example, in embodiments, the present disclosure provides a stable aqueous pharmaceutical formulation comprising ABBV-8E12 at a concentration of about 100 mg/ml; histidine; a polysorbate, and sucrose. In embodiments, the polysorbate is selected from the group consisting of polysorbate 20 and polysorbate 80. In embodiments, the polysorbate is PS80. In embodiments, the present disclosure provides a stable aqueous pharmaceutical formulation comprising ABBV-8E12 at a concentration of about 100 mg/ml; histidine at a concentration of about 5 to 50 mM; polysorbate 80 at a concentration of 1 to 10 mg/ml; and sucrose at a concentration of about 50 to about 100 mg/ml.

In embodiments, the present disclosure provides a stable aqueous pharmaceutical composition comprising ABBV-8E12, a surfactant, a buffer system comprising at least two buffers, and optionally an antioxidant, in which the concentration of ABBV-8E12 is about 100 mg/ml. In embodiments, the buffer may be selected from a combination of two or more buffers, such as acetate, citrate, gluconate, phosphate, succinate, histidine, tartrate, maleate, adipic acid, lactic acid, or trishydroxymethylaminomethane (tris). In embodiments, the buffer system is a citrate-phosphate buffer system.

In the foregoing embodiments comprising a buffer or combination of buffers, the buffer may be present at a concentration from about 5 mM to about 50 mM, such as from about 10 to about 40 mM, or from about 20 to about 30 mM, or about 25 mM. In the foregoing embodiments comprising a buffer or combination of buffers, the buffer may be selected from the group consisting of acetate, citrate, gluconate, phosphate, succinate, histidine, tartrate, maleate, adipic acid, lactic acid, or trishydroxymethylaminomethane (tris).

In the foregoing embodiments, the pH of the compositions may be from about 5 to about 7, such as from about 5.5 to about 6.5, or about 6.1.

In the foregoing embodiments, the surfactant may be present in an amount of from about 0.1 to about 10 mg/mL, such as from about 0.3 to about 5 mg/mL, or from about 0.5 to about 1.5 mg/mL, or about 1 mg/mL. In the foregoing embodiments, the surfactant may be a polysorbate. In embodiments, the polysorbate may be polysorbate 20 or polysorbate 80. In embodiments, the polysorbate may be polysorbate 80.

In the foregoing embodiments, the composition may comprise at least one additional excipient. In the foregoing embodiments, the excipient may be a stabilizer. In the foregoing embodiments, the at least one additional excipient is selected from the group consisting of a salt, a sugar, or a polyol. In the foregoing embodiments the at least one additional excipient may be a salt, and the salt may be present in an amount of from about 3 to about 20 mg/ml, such as from about 5 to about 10 mg/ml, or from about 8 to about 10 mg/ml, or about 8 mg/ml. In the foregoing embodiments, the at least one additional excipient may be a polyol, and the polyol may be present in an amount of from about 30 to about 50 mg/ml, or from about 35 to about 45 mg/ml, or from about 38 to about 43 mg/ml, or about 42 mg/ml. In the foregoing embodiments in which the at least one additional excipient is a polyol, the polyol may be a sugar alcohol, such as mannitol, sorbitol, or trehalose. In the foregoing embodiments, the at least one additional excipient may be a sugar, where the sugar may be present in an amount of from about 50 to about 100 mg/ml, such as from about 60 to about 90 mg/ml, or from about 70 to about 80 mg/ml, or about 75 mg/ml. In the foregoing embodiments in which the at least one additional excipient is a sugar, the sugar may be selected from the group consisting of sucrose, glucose, or dextrose. In embodiments, the sugar is sucrose. In the foregoing embodiments, the composition may comprise an antioxidant. In the foregoing embodiments, the antioxidant may be an amino acid. In embodiments, the amino acid is selected from the group consisting of glycine, arginine, and methionine. In embodiments, the amino acid is methionine. In the foregoing embodiments, the antioxidant may be present in an amount of from about 5 to about 15 mM, or from about 8 to about 12 mM, or about 10 mM.

In embodiments, the stable aqueous formulation according to any of the preceding embodiments is provided in a container. In embodiments, the container is a glass vial. In embodiments, the container is a polycarbonate vial. In embodiments, the container is a bag, such as an intravenous drip (IV) bag, which may be made, for example, of polyvinyl chloride or polyolefin. In embodiments, the container is an injection device, such as a microinfuser or a syringe. In embodiments, the composition according to the present disclosure may be diluted with, for example, saline solution. In embodiments, the composition may be diluted with saline solution within the container.

In any of the foregoing embodiments, ABBV-8E12 may be an antibody comprising a light chain comprising SEQ ID NO: 9, and a heavy chain comprising SEQ ID NO: 10.

In embodiments, the present disclosure provides a stable aqueous, buffered composition comprising an antibody at a concentration of about 100 mg/ml, wherein the antibody comprises a variable heavy chain that comprises a CDR1 sequence of SEQ ID NO: 6, a CDR2 sequence of SEQ ID NO:7, and a CDR3 sequence of SEQ ID NO: 8; and a variable light chain that comprises a CDR1 sequence of SEQ ID NO:2, a CDR2 sequence of SEQ ID NO: 3, and a CDR3 sequence of SEQ ID NO: 4; at least one buffer; an excipient; a surfactant; and optionally an anti-oxidant. In embodiments, the antibody comprises a light chain comprising an amino acid sequence as set forth in SEQ ID NO: 9 and a heavy chain comprising an amino acid sequence as set forth in SEQ ID NO: 10. In embodiments, the buffer is selected from the group consisting of histidine, phosphate, and succinate buffer. In embodiments, the buffer is histidine. In embodiments, the surfactant is a polysorbate. In embodiments, the surfactant is polysorbate 20 or polysorbate 80. In embodiments, the surfactant is polysorbate 80. In embodiments, the composition comprises an excipient selected from the group consisting of a salt, a sugar, and a polyol. In embodiments, the excipient is a salt, such as sodium chloride. In embodiments, the excipient is a polyol, such as a polyol selected from the group consisting of mannitol, sorbitol, and trehalose. In embodiments, the excipient is a sugar, such as a sugar selected from the group consisting of sucrose, glucose, and dextrose. In embodiments, the sugar is sucrose. In embodiments, the pH of the composition is from about 5 to about 7, such as from about 5.5 to about 6.5, or about 6.1.

In embodiments, the present disclosure provides a stable, aqueous, buffered composition comprising an antibody comprising a light chain of SEQ ID NO: 9 and a heavy chain of SEQ ID NO: 10, wherein the antibody is at a concentration of about 100 mg/ml; histidine; a polysorbate; an excipient; and optionally an anti-oxidant. In embodiments, the excipient is a salt, such as NaCl, at a concentration from about 3 to about 20 mg/ml. In embodiments, the excipient is a polyol, such as a polyol selected from the group consisting of mannitol, sorbitol, and trehalose, at a concentration of from about 30 to about 50 mg/ml. In embodiments, the excipient is a sugar, such as a sugar selected from the group consisting of sucrose, dextrose, and glucose, at a concentration of from about 50 to about 100 mg/ml. In embodiments, the polysorbate is polysorbate 80 or polysorbate 20. In embodiments, the polysorbate is polysorbate 80. In embodiments, the polysorbate is present at a concentration of 0.1 to about 10 mg/ml.

In embodiments, the present disclosure provides a stable aqueous, buffered composition comprising an antibody comprising a light chain of SEQ ID NO: 9 and a heavy chain of SEQ ID NO: 10, wherein the antibody is at a concentration of about 100 mg/ml; histidine; sucrose; and a polysorbate. In embodiments, histidine is present at a concentration of from about 5 mM to about 50 mM; sucrose is present at a concentration of from about 50 mg/ml to about 100 mg/ml; and the polysorbate is polysorbate 80 and is present at a concentration of from about 0.1 to about 10 mg/ml, such as from about 0.1 to about 1 mg/ml.

The invention is further illustrated in the following examples, which should not be construed as further limiting.

### Example 1: Formulation Screening Study

A large-scale formulation screening study was carried out to evaluate the stability of formulations of ABBV-8E12 at a 100 mg/ml protein concentration. The study evaluated formulations in 25 mM histidine, phosphate, or succinate buffer and in water. It also examined polysorbate 20 and polysorbate 80 as possible surfactants, and examined sucrose, sorbitol, and sodium chloride as possible isotonizers. Methionine was also examined as a possible antioxidant. In each formulation, 0.1% sodium azide was added as a preservative.

ABBV-8E12 in 50 mM histidine and 86 mg/ml sucrose, formulated at pH 6.08, was used as the bulk drug substance. Samples were prepared via 7-fold volume exchange diafiltration/ultrafiltration in 25 mM sodium citrate-phosphate buffer and subsequent diafiltration in three different buffer systems (histidine, phosphate, succinate) and WFI at pH 6.1. The protein content of these samples was adjusted to 120 mg/ml and sterile filtrated. Formulations were manually compounded into 96-well plates by mixing the protein solutions, excipient stock solutions, and sodium azide (0.1% as a preservative) to a target concentration of 100 mg/ml, as set forth in Table 1.

TABLE 1
Formulation Study in 96 well plates

| | Placement | | | | | | | | | | | | |
|---|---|---|---|---|---|---|---|---|---|---|---|---|---|
| | in 96 well | | | | | | | | | | | | |
| | plate | | ABBV- | | | | | | | | | | |
| Form # | Col | Row | 8E12 | Hist. | Phos. | Succ. | WFI | Sucrose | Sorbitol | NaCl | PS20 | PS80 | Met. |
| 1 | 1 | A | — | 25 mM | — | — | — | — | — | — | — | — | — |
| 2 | 1 | B | — | — | 25 mM | — | — | — | — | — | — | — | — |
| 3 | 1 | C | — | — | — | 25 mM | — | — | — | — | — | — | — |
| 4 | 1 | D | — | — | — | — | WFI | — | — | — | — | — | — |
| 5 | 2 | A | 100 mg/ml | 25 mM | — | — | — | — | — | — | 1 mg/ml | — | — |
| 6 | 2 | B | 100 mg/ml | 25 mM | — | — | — | 75 mg/ml | — | — | 1 mg/ml | — | — |
| 7 | 2 | C | 100 mg/ml | 25 mM | — | — | — | — | 42 mg/ml | — | 1 mg/ml | — | — |
| 8 | 2 | D | 100 mg/ml | 25 mM | — | — | — | — | — | 9 mg/ml | 1 mg/ml | — | — |
| 9 | 2 | E | 100 mg/ml | — | — | 25 mM | — | — | — | — | 1 mg/ml | — | — |
| 10 | 2 | F | 100 mg/ml | — | — | 25 mM | — | 75 mg/ml | — | — | 1 mg/ml | — | — |
| 11 | 2 | G | 100 mg/ml | — | — | 25 mM | — | — | 42 mg/ml | — | 1 mg/ml | — | — |
| 12 | 2 | H | 100 mg/ml | — | — | 25 mM | — | — | — | 9 mg/ml | 1 mg/ml | — | — |
| 13 | 3 | A | 100 mg/ml | — | 25 mM | — | — | — | — | — | 1 mg/ml | — | — |
| 14 | 3 | B | 100 mg/ml | — | 25 mM | — | — | 75 mg/ml | — | — | 1 mg/ml | — | — |
| 15 | 3 | C | 100 mg/ml | — | 25 mM | — | — | — | 42 mg/ml | — | 1 mg/ml | — | — |
| 16 | 3 | D | 100 mg/ml | — | 25 mM | — | — | — | — | 9 mg/ml | 1 mg/ml | — | — |
| 17 | 3 | E | 100 mg/ml | — | — | — | WFI | — | — | — | 1 mg/ml | — | — |
| 18 | 3 | F | 100 mg/ml | — | — | — | WFI | 75 mg/ml | — | — | 1 mg/ml | — | — |
| 19 | 3 | G | 100 mg/ml | — | — | — | WFI | — | 42 mg/ml | — | 1 mg/ml | — | — |
| 20 | 3 | H | 100 mg/ml | — | — | — | WFI | — | — | 9 mg/ml | 1 mg/ml | — | — |
| 21 | 4 | A | 100 mg/ml | 25 mM | — | — | — | — | — | — | — | 1 mg/ml | — |
| 22 | 4 | B | 100 mg/ml | 25 mM | — | — | — | 75 mg/ml | — | — | — | 1 mg/ml | — |
| 23 | 4 | C | 100 mg/ml | 25 mM | — | — | — | — | 42 mg/ml | — | — | 1 mg/ml | — |
| 24 | 4 | D | 100 mg/ml | 25 mM | — | — | — | — | — | 9 mg/ml | — | 1 mg/ml | — |
| 25 | 4 | E | 100 mg/ml | — | — | 25 mM | — | — | — | — | — | 1 mg/ml | — |
| 26 | 4 | F | 100 mg/ml | — | — | 25 mM | — | 75 mg/ml | — | — | — | 1 mg/ml | — |
| 27 | 4 | G | 100 mg/ml | — | — | 25 mM | — | — | 42 mg/ml | — | — | 1 mg/ml | — |
| 28 | 4 | H | 100 mg/ml | — | — | 25 mM | — | — | — | 9 mg/ml | — | 1 mg/ml | — |
| 29 | 5 | A | 100 mg/ml | — | 25 mM | — | — | — | — | — | — | 1 mg/ml | — |
| 30 | 5 | B | 100 mg/ml | — | 25 mM | — | — | 75 mg/ml | — | — | — | 1 mg/ml | — |
| 31 | 5 | C | 100 mg/ml | — | 25 mM | — | — | — | 42 mg/ml | — | — | 1 mg/ml | — |
| 32 | 5 | D | 100 mg/ml | — | 25 mM | — | — | — | — | 9 mg/ml | — | 1 mg/ml | — |
| 33 | 5 | E | 100 mg/ml | — | — | — | WFI | — | — | — | — | 1 mg/ml | — |
| 34 | 5 | F | 100 mg/ml | — | — | — | WFI | 75 mg/ml | — | — | — | 1 mg/ml | — |
| 35 | 5 | G | 100 mg/ml | — | — | — | WFI | — | 42 mg/ml | — | — | 1 mg/ml | — |
| 36 | 5 | H | 100 mg/ml | — | — | — | WFI | — | — | 9 mg/ml | — | 1 mg/ml | — |
| 37 | 6 | A | 100 mg/ml | 25 mM | — | — | — | — | — | — | — | 1 mg/ml | 10 mM |
| 38 | 6 | B | 100 mg/ml | 25 mM | — | — | — | 75 mg/ml | — | — | — | 1 mg/ml | 10 mM |
| 39 | 6 | C | 100 mg/ml | 25 mM | — | — | — | — | 42 mg/ml | — | — | 1 mg/ml | 10 mM |
| 40 | 6 | D | 100 mg/ml | 25 mM | — | — | — | — | — | 9 mg/ml | — | 1 mg/ml | 10 mM |
| 41 | 6 | E | 100 mg/ml | — | — | 25 mM | — | — | — | — | — | 1 mg/ml | 10 mM |
| 42 | 6 | F | 100 mg/ml | — | — | 25 mM | — | 75 mg/ml | — | — | — | 1 mg/ml | 10 mM |
| 43 | 6 | G | 100 mg/ml | — | — | 25 mM | — | — | 42 mg/ml | — | — | 1 mg/ml | 10 mM |
| 44 | 6 | H | 100 mg/ml | — | — | 25 mM | — | — | — | 9 mg/ml | — | 1 mg/ml | 10 mM |
| 45 | 7 | A | 100 mg/ml | — | 25 mM | — | — | — | — | — | — | 1 mg/ml | 10 mM |
| 46 | 7 | B | 100 mg/ml | — | 25 mM | — | — | 75 mg/ml | — | — | — | 1 mg/ml | 10 mM |
| 47 | 7 | C | 100 mg/ml | — | 25 mM | — | — | — | 42 mg/ml | — | — | 1 mg/ml | 10 mM |
| 48 | 7 | D | 100 mg/ml | — | 25 mM | — | — | — | — | 9 mg/ml | — | 1 mg/ml | 10 mM |
| 49 | 7 | E | 100 mg/ml | — | — | — | WFI | — | — | — | — | 1 mg/ml | 10 mM |
| 50 | 7 | F | 100 mg/ml | — | — | — | WFI | 75 mg/ml | — | — | — | 1 mg/ml | 10 mM |
| 51 | 7 | G | 100 mg/ml | — | — | — | WFI | — | 42 mg/ml | — | — | 1 mg/ml | 10 mM |
| 52 | 7 | H | 100 mg/ml | — | — | — | WFI | — | — | 9 mg/ml | — | 1 mg/ml | 10 mM |
| 53 | 8 | A | 100 mg/ml | 25 mM | — | — | — | — | — | — | — | — | — |
| 54 | 8 | B | 100 mg/ml | 25 mM | — | — | — | 75 mg/ml | — | — | — | — | — |
| 55 | 8 | C | 100 mg/ml | 25 mM | — | — | — | — | 42 mg/ml | — | — | — | — |
| 56 | 8 | D | 100 mg/ml | 25 mM | — | — | — | — | — | 9 mg/ml | — | — | — |
| 57 | 8 | E | 100 mg/ml | — | — | 25 mM | — | — | — | — | — | — | — |
| 58 | 8 | F | 100 mg/ml | — | — | 25 mM | — | 75 mg/ml | — | — | — | — | — |
| 59 | 8 | G | 100 mg/ml | — | — | 25 mM | — | — | 42 mg/ml | — | — | — | — |
| 60 | 8 | H | 100 mg/ml | — | — | 25 mM | — | — | — | 9 mg/ml | — | — | — |
| 61 | 9 | A | 100 mg/ml | — | 25 mM | — | — | — | — | — | — | — | — |
| 62 | 9 | B | 100 mg/ml | — | 25 mM | — | — | 75 mg/ml | — | — | — | — | — |
| 63 | 9 | C | 100 mg/ml | — | 25 mM | — | — | — | 42 mg/ml | — | — | — | — |
| 64 | 9 | D | 100 mg/ml | — | 25 mM | — | — | — | — | 9 mg/ml | — | — | — |
| 65 | 9 | E | 100 mg/ml | — | — | — | WFI | — | — | — | — | — | — |
| 66 | 9 | F | 100 mg/ml | — | — | — | WFI | 75 mg/ml | — | — | — | — | — |
| 67 | 9 | G | 100 mg/ml | — | — | — | WFI | — | 42 mg/ml | — | — | — | — |
| 68 | 9 | H | 100 mg/ml | — | — | — | WFI | — | — | 9 mg/ml | — | — | — |

Samples were stressed by four freeze/thaw cycles, shaking, and incubation at three different temperatures (5° C., 25° C., and 40° C.) for up to 12 weeks, as discussed in more detail below.

Analytical Block 1: Freeze-Thaw Conditions

In analytical block 1 (freeze-thaw stress), samples were frozen at −80° C. The frozen samples were then thawed at room temperature until all the ice had thawed. The freeze/thaw cycle was repeated three times for each sample (for a total of four freeze/thaw cycles).

Analytical Block 2: Mechanical Stress

Following Analytical Block 1, mechanical stress was applied in Analytical Block 2 by shaking the samples at 1200 rpm for three days at 20° C.

Analytical Block 3: Temperature

Following analytical block 2, the samples were aliquoted to 3×384 well plates as set forth in Table 2 for storage at 5° C., 25° C., and 40° C. Samples were stored for one week at 40° C., three weeks at 25° C. and 40° C., and 12 weeks at 5° C. and 25° C. More specifically, samples stored for one week at 40° C. were pulled at 7 days; samples stored for three weeks at 25° C. were pulled at 21 days; samples stored for three weeks at 40° C. were pulled at 24 days; samples stored for 12 weeks at 5° C. and 25° C. were pulled after 75 days. Any deviations from predefined pull points were considered non-critical.

TABLE 2
Formulation Study in 384 well plates

| | Placement | | | | | | | | | | | | |
|---|---|---|---|---|---|---|---|---|---|---|---|---|---|
| | in 384 well | | | | | | | | | | | | |
| | plate | | ABBV- | Buffer | | | | Stabilizer | | | Surfactant | | |
| Form. # | Col | Row | 8E12 | Hist. | Phos. | Succ. | WFI | Sucrose | Sorbitol | NaCl | PS20 | PS80 | Met. |
| 1 | 1 | A | — | 25 mM | — | — | — | — | — | — | — | — | — |
| 2 | 1 | C | — | — | 25 mM | | — | — | — | — | — | — | — |
| 3 | 1 | E | — | — | — | 25 mM | — | — | — | — | — | — | — |
| 4 | 1 | G | — | — | — | — | WFI | — | — | — | — | — | — |
| 5 | 3 | A | 100 mg/ml | 25 mM | — | — | — | — | — | — | 1 mg/ml | — | — |
| 6 | 3 | C | 100 mg/ml | 25 mM | — | — | — | 75 mg/ml | — | — | 1 mg/ml | — | — |
| 7 | 3 | E | 100 mg/ml | 25 mM | — | — | — | — | 42 mg/ml | — | 1 mg/ml | — | — |
| 8 | 3 | G | 100 mg/ml | 25 mM | — | — | — | — | — | 9 mg/ml | 1 mg/ml | — | — |
| 9 | 3 | I | 100 mg/ml | — | — | 25 mM | — | — | — | — | 1 mg/ml | — | — |
| 10 | 3 | K | 100 mg/ml | — | — | 25 mM | — | 75 mg/ml | — | — | 1 mg/ml | — | — |
| 11 | 3 | M | 100 mg/ml | — | — | 25 mM | — | — | 42 mg/ml | — | 1 mg/ml | — | — |
| 12 | 3 | O | 100 mg/ml | — | — | 25 mM | — | — | — | 9 mg/ml | 1 mg/ml | — | — |
| 13 | 5 | A | 100 mg/ml | — | 25 mM | — | — | — | — | — | 1 mg/ml | — | — |
| 14 | 5 | C | 100 mg/ml | — | 25 mM | | — | 75 mg/ml | — | — | 1 mg/ml | — | — |
| 15 | 5 | E | 100 mg/ml | — | 25 mM | — | — | — | 42 mg/ml | — | 1 mg/ml | — | — |
| 16 | 5 | G | 100 mg/ml | — | 25 mM | — | — | — | — | 9 mg/ml | 1 mg/ml | — | — |
| 17 | 5 | I | 100 mg/ml | — | — | — | WFI | — | — | — | 1 mg/ml | — | — |
| 18 | 5 | K | 100 mg/ml | — | — | — | WFI | 75 mg/ml | — | — | 1 mg/ml | — | — |
| 19 | 5 | M | 100 mg/ml | — | — | — | WFI | — | 42 mg/ml | — | 1 mg/ml | — | — |
| 20 | 5 | O | 100 mg/ml | — | — | — | WFI | — | — | 9 mg/ml | 1 mg/ml | — | — |
| 21 | 7 | A | 100 mg/ml | 25 mM | — | — | — | — | — | — | — | 1 mg/ml | — |
| 22 | 7 | C | 100 mg/ml | 25 mM | — | | — | 75 mg/ml | — | — | — | 1 mg/ml | — |
| 23 | 7 | E | 100 mg/ml | 25 mM | — | — | — | — | 42 mg/ml | — | — | 1 mg/ml | — |
| 24 | 7 | G | 100 mg/ml | 25 mM | — | — | — | — | — | 9 mg/ml | — | 1 mg/ml | — |
| 25 | 7 | I | 100 mg/ml | — | — | 25 mM | — | — | — | — | — | 1 mg/ml | — |
| 26 | 7 | K | 100 mg/ml | — | — | 25 mM | — | 75 mg/ml | — | — | — | 1 mg/ml | — |
| 27 | 7 | M | 100 mg/ml | — | — | 25 mM | — | — | 42 mg/ml | — | — | 1 mg/ml | — |
| 28 | 7 | O | 100 mg/ml | — | — | 25 mM | — | — | — | 9 mg/ml | — | 1 mg/ml | — |
| 29 | 9 | A | 100 mg/ml | — | 25 mM | — | — | — | — | — | — | 1 mg/ml | — |
| 30 | 9 | C | 100 mg/ml | — | 25 mM | — | — | 75 mg/ml | — | — | — | 1 mg/ml | — |
| 31 | 9 | E | 100 mg/ml | — | 25 mM | — | — | — | 42 mg/ml | — | — | 1 mg/ml | — |
| 32 | 9 | G | 100 mg/ml | — | 25 mM | — | — | — | — | 9 mg/ml | — | 1 mg/ml | — |
| 33 | 9 | I | 100 mg/ml | — | — | — | WFI | — | — | — | — | 1 mg/ml | — |
| 34 | 9 | K | 100 mg/ml | — | — | — | WFI | 75 mg/ml | — | — | — | 1 mg/ml | — |
| 35 | 9 | M | 100 mg/ml | — | — | — | WFI | — | 42 mg/ml | — | — | 1 mg/ml | — |
| 36 | 9 | O | 100 mg/ml | — | — | — | WFI | — | — | 9 mg/ml | — | 1 mg/ml | — |
| 37 | 11 | A | 100 mg/ml | 25 mM | — | — | — | — | — | — | — | 1 mg/ml | 10 mM |
| 38 | 11 | C | 100 mg/ml | 25 mM | — | | — | 75 mg/ml | — | — | — | 1 mg/ml | 10 mM |
| 39 | 11 | E | 100 mg/ml | 25 mM | — | — | — | — | 42 mg/ml | — | — | 1 mg/ml | 10 mM |
| 40 | 11 | G | 100 mg/ml | 25 mM | — | — | — | — | — | 9 mg/ml | — | 1 mg/ml | 10 mM |
| 41 | 11 | I | 100 mg/ml | — | — | 25 mM | — | — | — | — | — | 1 mg/ml | 10 mM |
| 42 | 11 | K | 100 mg/ml | — | — | 25 mM | — | 75 mg/ml | — | — | — | 1 mg/ml | 10 mM |
| 43 | 11 | M | 100 mg/ml | — | — | 25 mM | — | — | 42 mg/ml | — | — | 1 mg/ml | 10 mM |
| 44 | 11 | O | 100 mg/ml | — | — | 25 mM | — | — | — | 9 mg/ml | — | 1 mg/ml | 10 mM |
| 45 | 13 | A | 100 mg/ml | — | 25 mM | — | — | — | — | — | — | 1 mg/ml | 10 mM |
| 46 | 13 | C | 100 mg/ml | — | 25 mM | | — | 75 mg/ml | — | — | — | 1 mg/ml | 10 mM |
| 37 | 13 | E | 100 mg/ml | — | 25 mM | — | — | — | 42 mg/ml | — | — | 1 mg/ml | 10 mM |
| 48 | 13 | G | 100 mg/ml | — | 25 mM | — | — | — | — | 9 mg/ml | — | 1 mg/ml | 10 mM |
| 49 | 13 | I | 100 mg/ml | — | — | — | WFI | — | — | — | — | 1 mg/ml | 10 mM |
| 50 | 13 | K | 100 mg/ml | — | — | — | WFI | 75 mg/ml | — | — | — | 1 mg/ml | 10 mM |
| 51 | 13 | M | 100 mg/ml | — | — | — | WFI | — | 42 mg/ml | — | — | 1 mg/ml | 10 mM |
| 52 | 13 | O | 100 mg/ml | — | — | — | WFI | — | — | 9 mg/ml | — | 1 mg/ml | 10 mM |
| 53 | 15 | A | 100 mg/ml | 25 mM | — | — | — | — | — | — | — | — | — |
| 54 | 15 | C | 100 mg/ml | 25 mM | — | | — | 75 mg/ml | — | — | — | — | — |
| 55 | 15 | E | 100 mg/ml | 25 mM | — | — | — | — | 42 mg/ml | — | — | — | — |
| 56 | 15 | G | 100 mg/ml | 25 mM | — | — | — | — | — | 9 mg/ml | — | — | — |
| 57 | 15 | I | 100 mg/ml | — | — | 25 mM | — | — | — | — | — | — | — |
| 58 | 15 | K | 100 mg/ml | — | — | 25 mM | — | 75 mg/ml | — | — | — | — | — |
| 59 | 15 | M | 100 mg/ml | — | — | 25 mM | — | — | 42 mg/ml | — | — | — | — |
| 60 | 15 | O | 100 mg/ml | — | — | 25 mM | — | — | — | 9 mg/ml | — | — | — |
| 61 | 17 | A | 100 mg/ml | — | 25 mM | — | — | — | — | — | — | — | — |
| 62 | 17 | C | 100 mg/ml | — | 25 mM | | — | 75 mg/ml | — | — | — | — | — |
| 63 | 17 | E | 100 mg/ml | — | 25 mM | — | — | — | 42 mg/ml | — | — | — | — |
| 64 | 17 | G | 100 mg/ml | — | 25 mM | — | — | — | — | 9 mg/ml | — | — | — |
| 65 | 17 | I | 100 mg/ml | — | — | — | WFI | — | — | — | — | — | — |
| 66 | 17 | K | 100 mg/ml | — | — | — | WFI | 75 mg/ml | — | — | — | — | — |
| 67 | 17 | M | 100 mg/ml | — | — | — | WFI | — | 42 mg/ml | — | — | — | — |
| 68 | 17 | O | 100 mg/ml | — | — | — | WFI | — | — | 9 mg/ml | — | — | — |

Turbidity (Clarity and Opalescence)

Absorbance values at 700, 900, and 975 nm were measured for each analytical block (freeze/thaw, mechanical stress, temperature at 5° C., 25° C., and 40° C.) using a Tecan infinite M1000 Pro plate reader and used for the following turbidity calculation. The mathematical model used for converting absorption to turbidity units (NTU) is based on formazin turbidity standards. Briefly, the fill height of each well is recorded by subtraction of Abs900 value from Abs975 value, divided by the difference of these two wavelengths at 1 cm path length:

$$Fill Height = \frac{( Abs 975 - Abs 900 )}{0.1791}$$

Abs700 values are normalized by subtraction of plate specific values for 96 well (0.036) or 384 well plates (0.038): (Abs700—plate specific normalization factor). By division of the normalized Abs700 values through the calculated fill height, the Abs700 value is normalized for its fill height (Abs700norm/fill height). Multiplication of this normalized Abs700 value with 1248 results in the specific NTU value for each well: (Abs700norm*1248). The data is reported as calculated NTUs, but plate-reader based absorbance measurements and nephelometic turbidity determinations use different detection methods, and thus a direct correlation to nephelometric turbidity units is not possible.

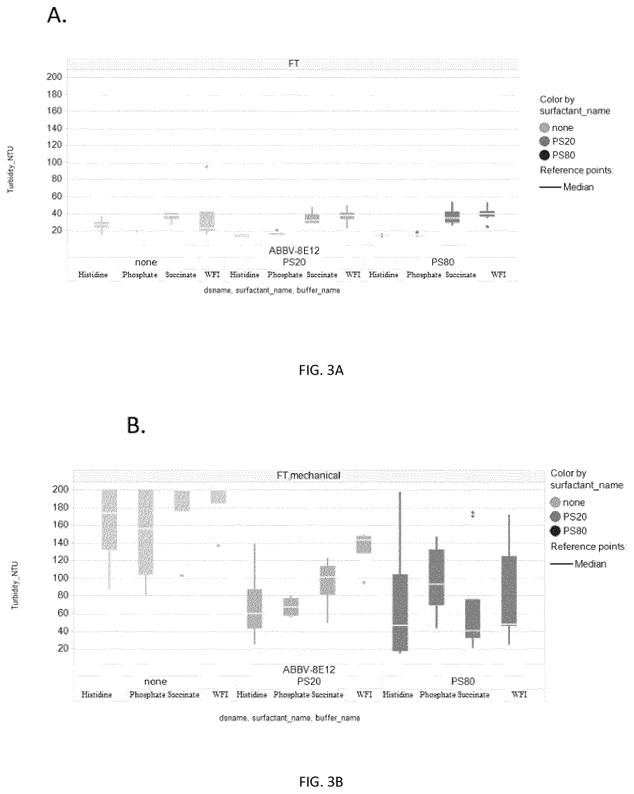

The turbidity of ABBV-8E12 formulations in 96 well plates (Table 1) following freeze/thaw and following freeze/thaw and mechanical stress are shown below in Table 3 and set forth in FIGS. 3A and B. The application of mechanical stress leads to higher turbidity, especially for samples formulated without polysorbate 20 or polysorbate 80 (i.e., the samples in Col. 8 and Col. 9). As shown in FIG. 3B and Table 3, most samples formulated without polysorbate 20 or polysorbate 80 have a turbidity value>200NTU after application of mechanical stress.

TABLE 3
Analytical Block 1 (Freeze/Thaw) and Analytical Block
2 (Mechanical Stress) Turbidity in calculated NTUs

| | | |
|---|---|---|
| Placement | | Turbidity after |
| in 96 well | Turbidity after | freeze/thaw and |
| plate | freeze/thaw | mechanical stress |

| Form # | Col. | Row | (NTU) | (NTU) |
|---|---|---|---|---|
| 1 | 1 | A | 6 | 1 |
| 2 | 1 | B | 2 | 3 |
| 3 | 1 | C | 5 | 6 |
| 4 | 1 | D | 3 | 3 |
| 5 | 2 | A | 15 | 25 |
| 6 | 2 | B | 13 | 71 |
| 7 | 2 | C | 16 | 139 |
| 8 | 2 | D | 13 | 49 |
| 9 | 2 | E | 29 | 49 |
| 10 | 2 | F | 36 | 92 |
| 11 | 2 | G | 48 | 111 |
| 12 | 2 | H | 29 | 123 |
| 13 | 3 | A | 15 | 58 |
| 14 | 3 | B | 14 | 55 |
| 15 | 3 | C | 21 | 80 |
| 16 | 3 | D | 15 | 77 |
| 17 | 3 | E | 37 | 139 |
| 18 | 3 | F | 38 | 95 |
| 19 | 3 | G | 50 | 148 |
| 20 | 3 | H | 23 | 149 |
| 21 | 4 | A | 15 | 42 |
| 22 | 4 | B | 13 | 15 |
| 23 | 4 | C | 17 | 18 |
| 24 | 4 | D | 12 | 97 |
| 25 | 4 | E | 26 | 174 |
| 26 | 4 | F | 33 | 34 |
| 27 | 4 | G | 43 | 41 |
| 28 | 4 | H | 26 | 21 |
| 29 | 5 | A | 15 | 73 |
| 30 | 5 | B | 14 | 77 |
| 31 | 5 | C | 15 | 143 |
| 32 | 5 | D | 14 | 43 |
| 33 | 5 | E | 37 | 137 |
| 34 | 5 | F | 40 | 43 |
| 35 | 5 | G | 50 | 47 |
| 36 | 5 | H | 25 | 172 |
| 37 | 6 | A | 15 | 51 |
| 38 | 6 | B | 14 | 16 |
| 39 | 6 | C | 16 | 128 |
| 40 | 6 | D | 14 | 198 |
| 41 | 6 | E | 31 | 170 |
| 42 | 6 | F | 37 | 40 |
| 43 | 6 | G | 54 | 45 |
| 44 | 6 | H | 42 | 28 |
| 45 | 7 | A | 14 | 129 |
| 46 | 7 | B | 13 | 59 |
| 47 | 7 | C | 18 | 147 |
| 48 | 7 | D | 15 | 109 |
| 49 | 7 | E | 39 | 47 |
| 50 | 7 | F | 41 | 121 |
| 51 | 7 | G | 53 | 49 |
| 52 | 7 | H | 34 | 25 |
| 53 | 8 | A | 37 | 263 |
| 54 | 8 | B | 26 | 534 |
| 55 | 8 | C | 28 | 147 |
| 56 | 8 | D | 15 | 87 |
| 57 | 8 | E | 36 | 368 |
| 58 | 8 | F | 42 | 103 |
| 59 | 8 | G | 40 | 997 |
| 60 | 8 | H | 27 | 1683 |
| 61 | 9 | A | 21 | 210 |
| 62 | 9 | B | 18 | 637 |
| 63 | 9 | C | 20 | 112 |
| 64 | 9 | D | 21 | 81 |
| 65 | 9 | E | 95 | 223 |
| 66 | 9 | F | 21 | 289 |
| 67 | 9 | G | 25 | 392 |
| 68 | 9 | H | 15 | 137 |

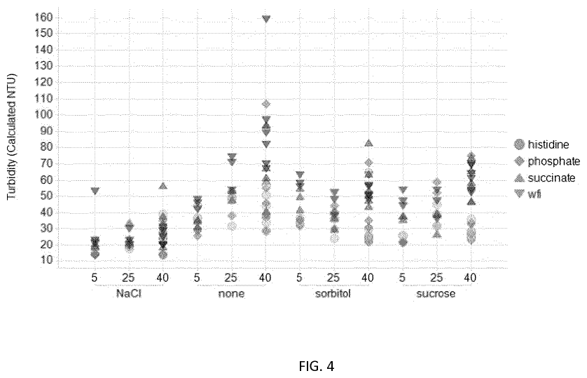

The turbidity of ABBV-8E12 formulations after temperature storage (5° C., 25° C., 40° C.) of the formulations in 384 well plates in Table 2 (polysorbate containing samples sorted by isotonizer and buffer) are shown in FIG. 4 and Table 4. As shown in FIG. 4 and Table 4, samples formulated in 25 mM histidine and phosphate buffer showed lower turbidities compared to succinate or WFI. Further, samples containing NaCl have the lowest turbidity values, while samples without stabilizer (“none”) have the highest turbidity values.

TABLE 4
Analytical Block 3 (Temperature) Turbidity (calculated NTUs)

| | Placement in 384 | | | | | |
|---|---|---|---|---|---|---|
| | well plate | | 1 Week | 3 weeks | | 12 weeks |
| Form. # | Col | Row | 40° C. | 25° C. | 40° C. | 5° C. |
| 1 | 1 | A | 5 | 9 | 9 | 11 |
| 2 | 1 | C | 4 | 6 | 5 | 10 |
| 3 | 1 | E | 2 | 2 | 4 | 16 |
| 4 | 1 | G | 0 | 4 | 0 | 5 |
| 5 | 3 | A | 29 | 32 | 34 | 36 |
| 6 | 3 | C | 25 | 45 | 29 | 22 |
| 7 | 3 | E | 22 | 24 | 25 | 33 |
| 8 | 3 | G | 14 | 20 | 25 | 15 |
| 9 | 3 | I | 41 | 48 | 62 | 32 |
| 10 | 3 | K | 47 | 27 | 47 | 38 |
| 11 | 3 | M | 48 | 30 | 51 | 42 |
| 12 | 3 | O | 19 | 24 | 38 | 21 |
| 13 | 5 | A | 29 | 38 | 39 | 26 |
| 14 | 5 | C | 23 | 32 | 27 | 22 |
| 15 | 5 | E | 22 | 30 | 26 | 32 |
| 16 | 5 | G | 15 | 19 | 28 | 14 |
| 17 | 5 | I | 70 | 54 | 159 | 42 |
| 18 | 5 | K | 52 | 36 | 61 | 47 |
| 19 | 5 | M | 48 | 38 | 50 | 56 |
| 20 | 5 | O | 20 | 20 | 25 | 23 |
| 21 | 7 | A | 38 | 48 | 51 | 44 |
| 22 | 7 | C | 25 | 32 | 28 | 26 |
| 23 | 7 | E | 24 | 40 | 26 | 34 |
| 24 | 7 | G | 14 | 19 | 28 | 23 |
| 25 | 7 | I | 60 | 54 | 94 | 35 |
| 26 | 7 | K | 57 | 39 | 74 | 36 |
| 27 | 7 | M | 44 | 37 | 64 | 50 |
| 28 | 7 | O | 23 | 22 | 34 | 19 |
| 29 | 9 | A | 37 | 50 | 55 | 30 |
| 30 | 9 | C | 27 | 59 | 33 | 21 |
| 31 | 9 | E | 24 | 37 | 35 | 35 |
| 32 | 9 | G | 14 | 19 | 35 | 14 |
| 33 | 9 | I | 67 | 74 | 97 | 46 |
| 34 | 9 | K | 54 | 47 | 64 | 54 |
| 35 | 9 | M | 56 | 48 | 51 | 58 |
| 36 | 9 | O | 20 | 23 | 29 | 53 |
| 37 | 11 | A | 44 | 49 | 57 | 37 |
| 38 | 11 | C | 26 | 39 | 36 | 26 |
| 39 | 11 | E | 30 | 36 | 65 | 37 |
| 40 | 11 | G | 24 | 18 | 39 | 23 |
| 41 | 11 | I | 68 | 54 | 94 | 36 |
| 42 | 11 | K | 59 | 40 | 66 | 38 |
| 43 | 11 | M | 54 | 41 | 83 | 55 |
| 44 | 11 | O | 26 | 34 | 57 | 20 |
| 45 | 13 | A | 46 | 53 | 107 | 29 |
| 46 | 13 | C | 34 | 52 | 75 | 22 |
| 47 | 13 | E | 31 | 44 | 71 | 36 |
| 48 | 13 | G | 21 | 20 | 27 | 15 |
| 49 | 13 | I | 82 | 71 | 89 | 48 |
| 50 | 13 | K | 70 | 54 | 69 | 44 |
| 51 | 13 | M | 63 | 52 | 57 | 63 |
| 52 | 13 | O | 29 | 30 | 31 | 21 |
| 53 | 15 | A | 665 | 281 | 615 | 176 |
| 54 | 15 | C | 658 | 258 | 646 | 141 |
| 55 | 15 | E | 97 | 104 | 106 | 75 |
| 56 | 15 | G | 40 | 38 | 51 | 29 |
| 57 | 15 | I | 350 | 387 | 324 | 177 |
| 58 | 15 | K | 122 | 99 | 97 | 68 |
| 59 | 15 | M | 542 | 368 | 344 | 420 |
| 60 | 15 | O | 458 | 136 | 397 | 531 |
| 61 | 17 | A | 236 | 236 | 215 | 167 |
| 62 | 17 | C | 421 | 547 | 365 | 279 |
| 63 | 17 | E | 57 | 81 | 60 | 64 |
| 64 | 17 | G | 41 | 35 | 54 | 27 |
| 65 | 17 | I | 222 | 181 | 221 | 66 |
| 66 | 17 | K | 242 | 270 | 247 | 98 |
| 67 | 17 | M | 330 | 550 | 358 | 164 |
| 68 | 17 | O | 225 | 162 | 281 | 62 |

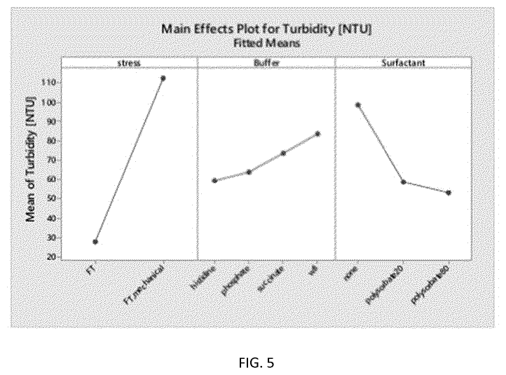

Statistical analysis reveals that the main effects for turbidity formation prior to temperature storage are stress, buffer type, and surfactant, as shown in FIG. 5, which provides a main effects plot of turbidity after application of freeze/thaw and mechanical stress. Turbidity increases through the application of mechanical stress, most pronounced for samples without surfactant. Samples formulated in histidine buffer have in general the lowest turbidities.

After temperature storage, as shown in FIG. 4, main effects for turbidity increase are buffer and isotonizer type, but also surfactant type.

Size Exclusion-UHPLC (SE-UHPLC): monomer, aggregates, fragments

SE-UHPLC was performed with 100 mg/ml ABBV-8E12 samples. To detect reversible aggregates, samples were diluted with WFI to 1 mg/ml and analyzed by SE-UHPLC after incubation at 40%/75% RH for 8 hours. Analysis of reversible aggregation was not performed at time TO.

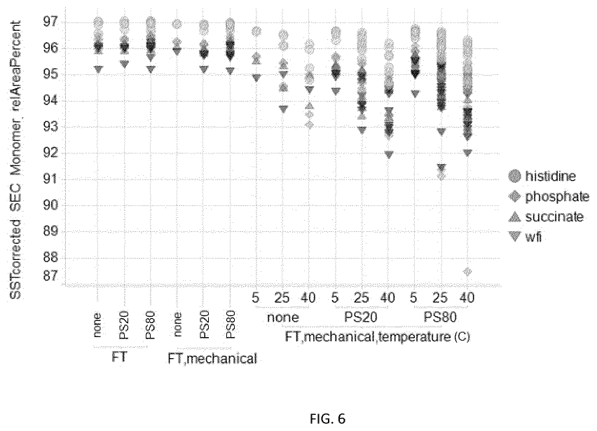

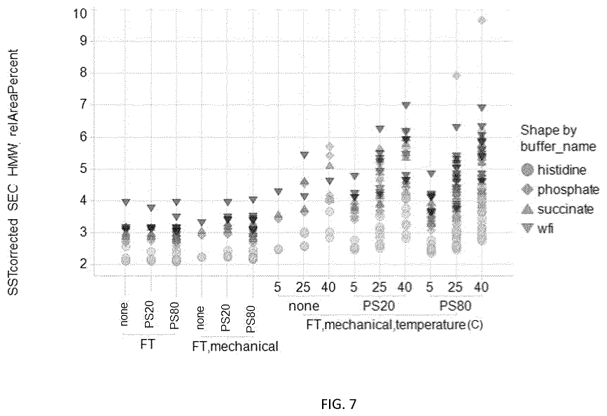

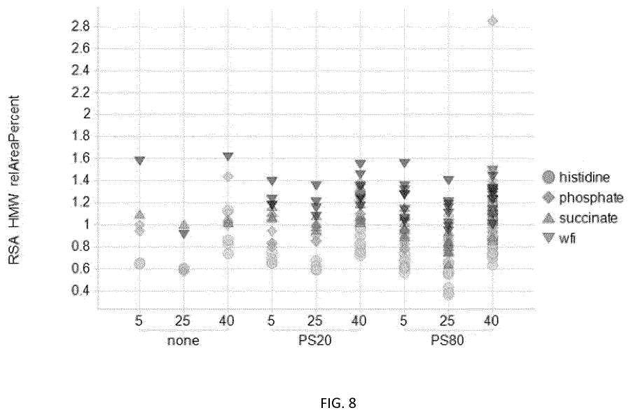

FIG. 6 shows monomer contents of all ABBV-8E12 samples, as measured by SE-UHPLC. Samples formulated in histidine buffer show highest monomer content, followed by samples formulated in phosphate or succinate buffer or water. Monomer loss mainly results in the formation of aggregates, as shown in FIG. 7.

Size Exclusion-UHPLC: Reversible aggregation

Measured reversible aggregation was below 1.6% after storage at 5° C., 25° C., and 40° C., as shown in FIG. 8. Only one sample formulated in phosphate buffer (containing sucrose, methionine, and polysorbate 80) for 21 days at 40° C. deviates.

Formulation Composition for ABBV-8E12

The results indicate the stabilizing effect of histidine as a buffer system. Compared to phosphate, succinate buffer, or water for injection (WFI, no buffer), the histidine containing samples maintain higher levels of monomer under almost all stress conditions.

The turbidity data clearly showed that a surfactant is required to maintain the colloidal stability of the formulation during mechanical stress. PS20 and PS80 were both suitable surfactants.

### Example 2: Long-Term Storage of ABBV-8E12 Formulations

The stability of a 100 mg/mL formulation of ABBV-8E12 was compared to the stability of a 20 mg/mL formulation of ABBV-8E12 using data from long term conditions (−70° C./−80° C.) and real time condition (5° C.).

TABLE 5
Formulations for Long-Term Storage Assessment

| | | | Time point |
|---|---|---|---|
| Formulation | Composition | Temperature | (month) |
| A | 20 mg/ml ABBV-8E12 | −70° C. | 0, 3, 6 |
| | 50 mM Histidine | 5° C. | 0, 1, 3, 6 |
| | 8.6% (w/v) Sucrose | | |
| | 0.02% (w/v) Polysorbate 20 | | |
| | pH 6.0 | | |
| B | 100 mg/ml ABBV-8E12 | −80° C. | 0, 1.5, 3, 6 |
| | 25 mM Histidine | 5° C. | 0, 1.5, 3, 6 |
| | 7.5% (w/v) Sucrose | | |
| | 0.1% (w/v) Polysorbate 80 | | |
| | pH 6.1 | | |

A batch comprising formulation A (ABBV-8E12 at 20 mg/ml), i.e., Formulation A-1, was compared to two batches comprising Formulation B (ABBV-8E12 at 100 mg/mL), i.e., Formulations B-1 and B-2. The data presented here demonstrates that the 100 mg/ml formulation, having a significantly higher protein concentration, demonstrated comparable stability over the reported storage period compared to the 20 mg/ml formulation.

Size-exclusion chromatography separates molecules according to their hydrodynamic radius. The stationary phase consists of gel-like particles with a defined pore size. Molecules larger than these pores elute first, whereas molecules smaller than these pores of the stationary phase take longer to migrate through the column and thus elute later. The detection is performed at 214 nm and the impurities (high molecular weight species and low molecular weight species) are determined as relative area in area-%.

CE-SDS determines the purity using capillary gel electrophoresis—sodium dodecyl sulfate (CE-SDS). In CE-SDS, samples are diluted in SDS sample buffer and electrokinetically injected onto an uncoated (non-treated) bare fused silica capillary which is filled with a replaceable SDS gel buffer containing 0.2% SDS. The sample proteins form a complex of same charge to size ratio with SDS allowing for separation to occur by size with smaller proteins migrating faster (or eluting earlier) than larger protein. The assay is performed under reducing and non-reducing conditions. Peak detection is based on the UV absorbance at 214 nm and quantitation is based on relative area percent.

TABLE 6
Long Term Storage of ABBV-8E12 Formulations (Protein Content, SE-HPLC)

| | | Comparability | | −70° C. /−80° C. | | | 5° C. | | |
|---|---|---|---|---|---|---|---|---|---|
| Test Item | | Criterion | Duration | A-1 | B-1 | B-2 | A-1 | B-1 | B-2 |
| A₂₈₀ (protein | | 20 ± 2/100 ± | Initial | 19.9 | 100 | 94 | 19.9 | 100 | 94 |
| content) (mg/mL) | | 10 | 1/1.5 | — | 101 | 95 | 19.9 | 101 | 96 |
| | | | 3 | 19.7 | 100 | n/a | 19.7 | 100 | n.a. |
| | | | 6 | 19.5 | n.a. | n.a. | 20.3 | n.a. | n.a. |
| SE- | Monomer | NLT 90.0 | Initial | 98.7 | 97.8 | 98.1 | 98.7 | 97.8 | 98.1 |
| HPLC | [%] | | 1/1.5 | — | 97.8 | 98.0 | 98.5 | 97.6 | 97.8 |
| | | | 3 | 98.7 | 97.8 | 98.1 | 98.5 | 97.4 | 97.7 |
| | | | 6 | 98.7 | n/a | n/a | 98.5 | n/a | n/a |
| | HMWS | NMT 5.0 | Initial | 1.3 | 2.2 | 1.9 | 1.3 | 2.2 | 1.9 |
| | [%] | | 1/1.5 | — | 2.2 | 1.9 | 1.3 | 2.2 | 1.9 |
| | | | 3 | 1.3 | 2.2 | 1.9 | 1.4 | 2.6 | 2.2 |
| | | | 6 | 1.3 | n/a | n/a | 1.5 | n/a | n/a |
| | LMWS | Report | Initial | <0.3 | <0.3 | <0.3 | <0.3 | <0.3 | <0.3 |
| | | Result | 1/1.5 | — | <0.3 | <0.3 | <0.3 | <0.3 | <0.3 |
| | | | 3 | <0.3 | <0.3 | <0.3 | <0.3 | <0.3 | <0.3 |
| | | | 6 | <0.3 | n/a | n/a | <0.3 | n/a | n/a |

— = not tested;
n/a = not available

TABLE 7
Long Term Storage of ABBV-8E12 Formulations (CE-SDS-R)

| | | Comparability | | −70° C./−80° C. | | | 5° C. | | |
|---|---|---|---|---|---|---|---|---|---|
| Test Item | | Criterion | Duration | A-1 | B-1 | B-2 | A-1 | B-1 | B-2 |
| CE- | Purity | NLT 90.0 | Initial | — | 97.9 | 97.9 | — | 97.9 | 97.9 |
| SDS- | [%] | | 1/1.5 | — | 97.9 | 97.5 | — | 97.8 | 97.5 |
| R | | | 3 | — | 97.7 | 97.9 | — | 97.7 | 97.8 |
| | | | 6 | — | n/a | n/a | — | n/a | n/a |
| | LMWS | To be | Initial | — | 1.2 | 1.3 | — | 1.2 | 1.3 |
| | % | monitored | 1/1.5 | — | 1.3 | 1.6 | — | 1.3 | 1.5 |
| | | | 3 | — | 1.3 | 1.2 | — | 1.3 | 1.3 |
| | | | 6 | — | n/a | n/a | — | n/a | n/a |
| | Late | To be | Initial | — | 0.9 | 0.8 | — | 0.9 | 0.8 |
| | migrating | monitored | 1/1.5 | — | 0.8 | 0.9 | — | 1.0 | 1.0 |
| | species % | | 3 | — | 1.0 | 0.9 | — | 1.0 | 1.0 |
| | | | 6 | — | n/a | n/a | — | n/a | n/a |

— = not tested;
n/a = not available

TABLE 8
Long Term Storage of ABBV-8E12 Formulations (CE-SDS-NR, pH)

| | | Comparability | | −70° C./−80° C. | | | 5° C. | | |
|---|---|---|---|---|---|---|---|---|---|
| Test Item | | Criterion | Duration | A-1 | B-1 | B-2 | A-1 | B-1 | B-2 |
| CE- | Purity | NLT 90.0 | Initial | — | 98.4 | 98.5 | — | 98.4 | 98.5 |
| SDS- | [%] | | 1/1.5 | — | 98.8 | 99.0 | — | 98.8 | 98.9 |
| NR | | | 3 | — | 98.4 | 98.6 | — | 98.4 | 98.5 |
| | | | 6 | — | n/a | n/a | — | n/a | n/a |
| | HMWS | Report | Initial | — | 0.5 | 0.5 | — | 0.5 | 0.5 |
| | % | Result | 1/1.5 | — | 0.3 | 0.2 | — | 0.3 | 0.3 |
| | | | 3 | — | 0.5 | 0.3 | — | 0.5 | 0.4 |
| | | | 6 | — | n/a | n/a | — | n/a | n/a |
| | LMWS | Report | Initial | — | 1.1 | 1.0 | — | 1.1 | 1.0 |
| | | Result | 1/1.5 | — | 0.9 | 0.8 | — | 0.9 | 0.9 |
| | | | 3 | — | 1.1 | 1.1 | — | 1.1 | 1.1 |
| | | | 6 | — | n/a | n/a | — | n/a | n/a |

| | | | | | | | | |
|---|---|---|---|---|---|---|---|---|
| pH | 5.5 to 6.6 | Initial | 6.0 | 6.2 | 6.1 | 6.0 | 6.2 | 6.1 |

| | | | | | | |
|---|---|---|---|---|---|---|
| 1/1.5 | n/a | 6.1 | 6.1 | 6.0 | 6.1 | 6.2 |
| 3 | 6.0 | 6.1 | 6.2 | 6.0 | 6.2 | 6.2 |
| 6 | 6.0 | 6.1 | n/a | 6.0 | 6.1 | n/a |

— = not tested;
n/a = not available

TABLE 9
Long-term Storage of ABBV-8E12 Formulations (Clarity, Color)

| | Comparability | | | −70° C./−80° C. | | | 5° C. | | |
|---|---|---|---|---|---|---|---|---|---|
| Test Item | Criterion | Duration | | A-1 | B-1 | B-2 | A-1 | B-1 | B-2 |
| Clarity | Report result | Initial | | <RS | ≤RS | ≤RS | <RS | ≤RS | ≤RS |
| (Reference | (target ≤ Ph. Eur. | | | III | IV | IV | III | IV | IV |
| suspension) | Reference | 1/1.5 | month | — | ≤RS | ≤RS | <RS | ≤RS | ≤RS |
| | Suspension | | | | IV | IV | III | IV | IV |
| | IV | 3 | month | <RS | ≤RS | ≤RS | <RS | ≤RS | ≤RS |
| | | | | III | IV | IV | III | IV | IV |
| | | 6 | month | <RS | ≤RS | n.a. | <RS | ≤RS | n.a. |
| | | | | III | IV | | III | IV | |
| Clarity | Report | Initial | | 8 | 22 | 22 | 8 | 22 | 22 |
| (NTU) | Result | 1/1.5 | month | — | 22 | 22 | 8 | 22 | 22 |
| | (Target ≤30 | 3 | month | 8 | 23 | 23 | 8 | 23 | 22 |
| | NTU) | 6 | month | 8 | n.a. | n.a. | 8 | n.a. | n.a. |
| Color | Less colored | Initial | | =B6 | ≤B5 | ≤B5 | =B6 | ≤B5 | ≤B5 |
| (B-Scale) | than or equal | 1/1.5 | month | — | ≤B4 | ≤B4 | =B6 | ≤B4 | ≤B4 |
| | to Reference | 3 | month | =B6 | ≤B5 | ≤B5 | =B6 | ≤B5 | ≤B5 |
| | solution B4 | 6 | month | =B6 | ≤B4 | n.a. | =B6 | ≤B5 | n.a. |

More detailed comparison of Formulation A and Formulation B batches show differences for clarity, color, protein content, and pH. These differences are due mainly to the differences in formulation, mainly the increased protein concentration. A slight increase in HMW (SE-HPLC) has been observed for all three batches stored at 5° C. These changes in HMW are small and are within the comparability criteria. All long-term stability (−70° C./−80° C.) and real time stability (5° C.) data are within release and stability criteria. Accordingly, the data presented here demonstrates that the 100 mg/ml formulation, while having a significantly higher protein concentration, demonstrated comparability over the reported storage period as compared to the 20 mg/ml formulation.

### Example 3: Properties of ABBV-8E12 Formulation

The concentration, pH, density (g/cm³), viscosity (mPas) and osmolality of Formulation B-1 were measured. The density and viscosity were determined at 20° C. The results are presented in Table 10.

TABLE 10
Properties of B1, measured after pooling

| Parameter | Value |
|---|---|
| Concentration (g/L) | 104 |
| pH | 6.15 |
| Density* (g/cm³) | 1.06 |
| Viscosity* (mPas) | 4.48 |
| Osmolality (mOsmol/kg) | 319 |

*Value was determined at 20° C.

### Example 4: Long Term Stability of ABBV-8E12 Formulations

The following example describes a study which examined the long-term storage stability of formulations having an ABBV-8E12 concentration of 100 mg/mL as compared to formulations having an ABBV-8E12 concentration of 20 mg/ml. The composition of the two formulations is described in Table 11.

TABLE 11
Formulations for Long-Term Stability Assessment

| | | | Time point |
|---|---|---|---|
| Formulation | Composition | Temperature | (month) |
| A | 20 mg/ml ABBV-8E12 | 4° C. | 0, 3, 6, 9, 12 |
| | 50 mM Histidine | 25° C. | 0, 1, 3, 6 |
| | 8.6% (w/v) Sucrose | 40° C. | 0, 1, 3 |
| | 0.02% (w/v) Polysorbate 20 | | |
| | pH 6.0 | | |
| B | 100 mg/mL ABBV-8E12 | 4° C. | 0, 1, 3, 6, 9 |
| | 3.88 mg/mL L-Histidine | 25° C. | 0, 1, 3, 6 |
| | 75.0 mg/mL Sucrose | 40° C. | 0, 1, 3 |
| | 1.00 mg/mL Polysorbate 80 | | |
| | Hydrochloric acid - | | |
| | Adjust to pH 6.1 | | |
| | 1 mL WFI | | |

One batch of Formulation A (A2) was compared against three batches of Formulation B (B3, B4, B5) at 4° C., 25° C., and at 40° C. In particular, formulation A2 was tested prior to long term storage and after 1 month (25° C., 40° C.), 3 months (4° C., 25° C., 40° C.), 6 months (4° C., 25° C.), 9 months (4° C.), and 12 months (4° C.). Formulations B3, B4, and B5 were tested prior to long term storage and after 1 month (4° C., 25° C., 40° C.), 3 months (4° C., 25° C., 40° C.), 6 months (4° C., 25° C.), and 9 months (4° C.).

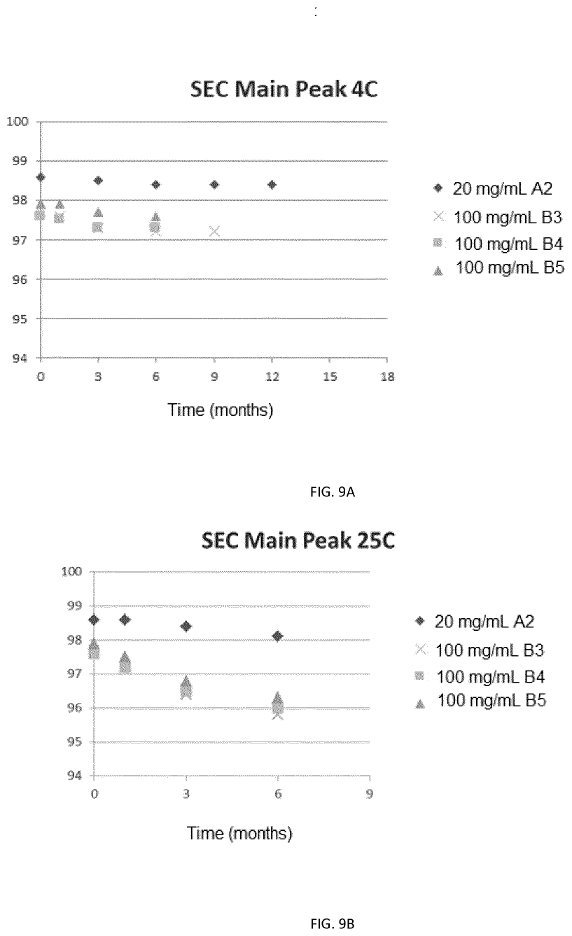

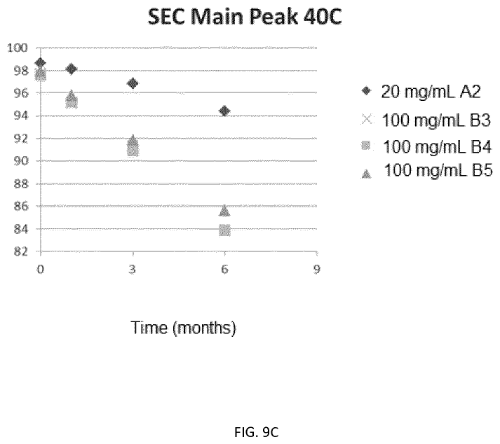

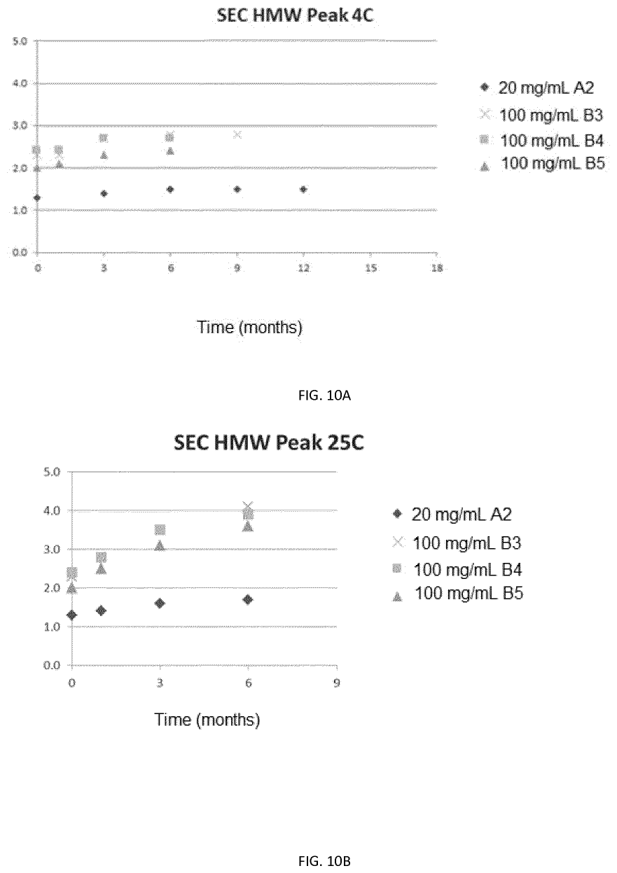

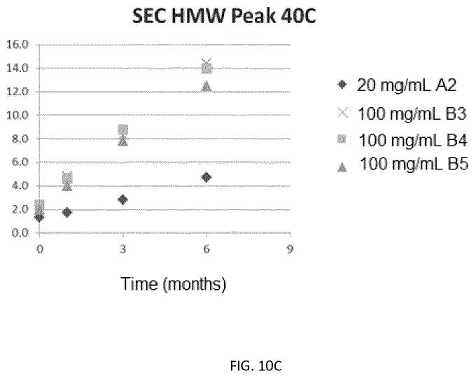

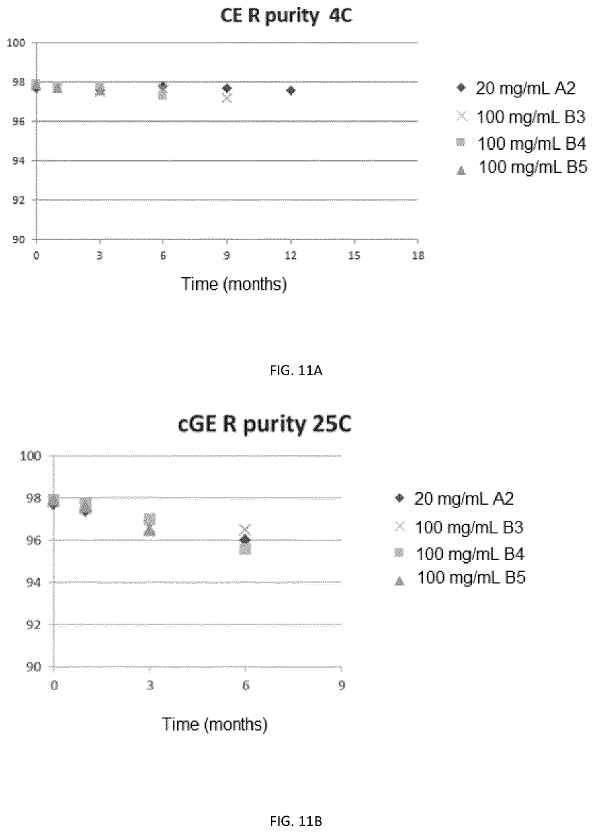

The SEC results for the samples are shown in FIGS. 9A-C and 10A-C. FIG. 9A-9C shows monomer contents of the four ABBV-8E12 samples; in particular, FIG. 9A shows SEC main peak data at 4° C., FIG. 9B shows SEC main peak data at 25° C., and FIG. 9C shows SEC main peak data at 40° C. FIG. 10A-10C show high molecular weight (HMW) aggregates as measured by SEC; in particular, FIG. 10A shows SEC HMW peak at 4° C., FIG. 10B shows SEC HMW peak data at 25° C., and FIG. 10C shows SEC HMW Peak data at 40° C.

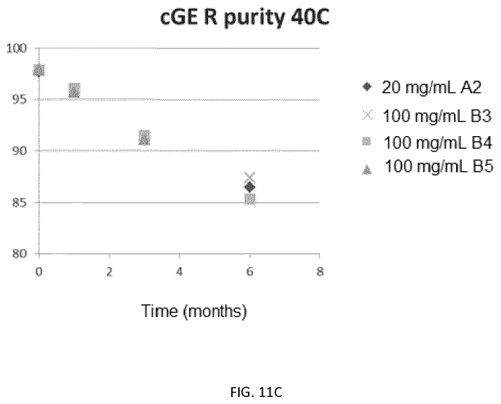

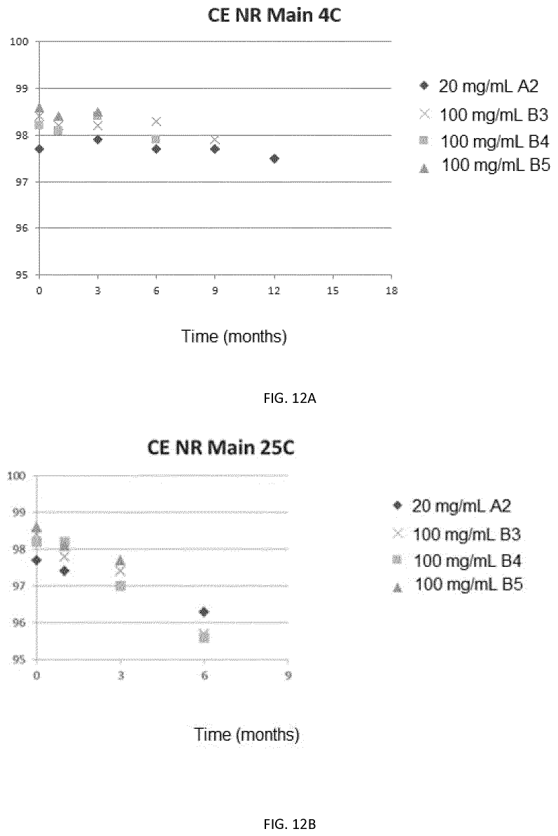

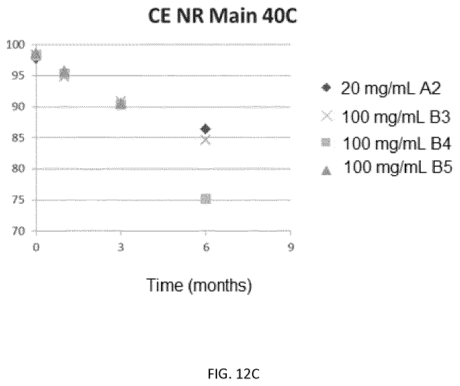

Capillary gel electrophoresis provides automated analysis of reduced (CE R) and non-reduced (CE NR) proteins by size to determine protein purity and/or heterogeneity. The results for the CE analysis are shown in FIGS. 11A-C(CE reduced % purity) and 12A-C(CE non-reduced % main).

The results of the long-term stability experiments show that 100 mg/ml formulations of ABBV-8E12 are stable when subjected to long-term storage.

It is understood that the foregoing detailed description and accompanying examples are merely illustrative and are not to be taken as limitations upon the scope of the present disclosure, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations and/or methods of use of the present disclosure, may be made without departing from the spirit and scope thereof. All publications, patents, and patent applications cited herein are hereby incorporated by reference in their entirety for all purposes.

| SEQ ID | |
|---|---|
| NO: | Sequence |
| 1 | DIVLTQSPDSLAVSLGERATISCRASQSVSTSRYSYIH |
| | WYQQKPGQPPKLLIKYASNLESGVPSRFSGSGSGTD |
| | FTLNIHPLEPEDFATYYCHHSWEIPLTFGQGTKLEIK |
| 2 | RASQSVSTSRYSYIH |
| 3 | YASNLES |
| 4 | HHSWEIPLT |
| 5 | EVKVVESGGGLVQPGGSMKLSCVVSGFTFSNYWVNW |
| | VRQAPGKGLEWVAQIRLKSDNYATHYEESVKGRFTIS |
| | RDDSKSSVYLQMNNLRAEDSGIYYCTNWEDYWGQGT |
| | TVTVSSASTKGPSVF |
| 6 | NYWVN |
| 7 | QIRLKSDNYATHYEESVKG |
| 8 | WEDY |
| 9 | DIVLTQSPDSLAVSLGERATISCRASQSVSTSRYSYIH |
| | WYQQKPGQPPKLLIKYASNLESGVPSRFSGSGSGTD |
| | FTLNIHPLEPEDFATYYCHHSWEIPLTFGQGTKLEIKR |
| | TVAAPSVFIFPPSDEQLKSGTASVVCLLNNFYPREAK |
| | VQWKVDNALQSGNSQESVTEQDSKDSTYSLSSTLTL |
| | SKADYEKHKVYACEVTHQGLSSPVTKSFNRGEC |
| 10 | EVKVVESGGGLVQPGGSMKLSCVVSGFTFSNYWVNW |
| | VRQAPGKGLEWVAQIRLKSDNYATHYEESVKGRFTIS |
| | RDDSKSSVYLQMNNLRAEDSGIYYCTNWEDYWGQGT |
| | TVTVSSASTKGPSVFPLAPCSRSTSESTAALGCLVKD |
| | YFPEPVTVSWNSGALTSGVHTFPAVLQSSGLYSLSSV |
| | VTVPSSSLGTKTYTCNVDHKPSNTKVDKRVESKYGPP |
| | CPPCPAPEFLGGPSVFLFPPKPKDTLMISRTPEVTCV |
| | VVDVSQEDPEVQFNWYVDGVEVHNAKTKPREEQFNS |
| | TYRVVSVLTVLHQDWLNGKEYKCKVSNKGLPSSIEKT |
| | ISKAKGQPREPQVYTLPPSQEEMTKNQVSLTCLVKGF |
| | YPSDIAVEWESNGQPENNYKTTPPVLDSDGSFFLYSR |
| | LTVDKSRWQEGNVFSCSVMHEALHNHYTQKSLSLSL |
| | GK |

1. A stable aqueous, buffered composition comprising:
an antibody at a concentration of about 100 mg/ml, wherein the antibody comprises a variable heavy chain that comprises a CDR1 sequence of SEQ ID NO: 6, a CDR2 sequence of SEQ ID NO:7, and a CDR3 sequence of SEQ ID NO: 8; and a variable light chain that comprises a CDR1 sequence of SEQ ID NO:2, a CDR2 sequence of SEQ ID NO: 3, and a CDR3 sequence of SEQ ID NO: 4;
at least one buffer;
an excipient;
a surfactant; and
optionally an anti-oxidant.

2. The composition according to claim 1, wherein the antibody comprises a light chain comprising an amino acid sequence as set forth in SEQ ID NO: 9 and a heavy chain comprising an amino acid sequence as set forth in SEQ ID NO: 10.

3. The composition according to any of the preceding claims, wherein the buffer is selected from the group consisting of histidine, phosphate, and succinate buffer.

4. The composition according to claim 3, wherein the buffer is histidine.

5. The composition according to any of the preceding claims, wherein the surfactant is a polysorbate.

6. The composition according to claim 5, wherein the surfactant is Polysorbate 80.

7. The composition according to any of the preceding claims, wherein the composition comprises an excipient selected from the group consisting of a salt, a sugar, and a polyol.

8. The composition according to claim 7, wherein the excipient is sodium chloride.

9. The composition according to claim 7, wherein the excipient is a polyol selected from the group consisting of mannitol, sorbitol, and trehalose.

10. The composition according to claim 7, wherein the excipient is a sugar selected from the group consisting of sucrose, glucose, and dextrose.

11. The composition according to any of the proceeding claims, wherein the pH is about 5 to about 7.

12. A stable aqueous, buffered composition comprising:
an antibody comprising a light chain of SEQ ID NO: 9 and a heavy chain of SEQ ID NO: 10, wherein the antibody is at a concentration of about 100 mg/ml;
histidine;
a polysorbate;
an excipient; and
optionally an anti-oxidant.

13. The composition according to claim 12, wherein the excipient is a salt at a concentration from about 3 to about 20 mg/ml.

14. The composition according to claim 13, wherein the salt is sodium chloride.

15. The composition according to claim 12, wherein the excipient is a polyol selected from the group consisting of mannitol, sorbitol, and trehalose at a concentration of from about 30 to about 50 mg/ml.

16. The composition according to claim 12, wherein the excipient is a sugar selected from the group consisting of sucrose, dextrose, and glucose at a concentration of from about 50 to about 100 mg/ml.

17. The composition according to claims 12-16, wherein the polysorbate is polysorbate 80 or polysorbate 20.

18. The composition according to claim 17, wherein the polysorbate is present at a concentration of 0.1 to about 10 mg/ml.

19. A stable aqueous, buffered composition comprising:
an antibody comprising a light chain of SEQ ID NO: 9 and a heavy chain of SEQ ID NO: 10, wherein the antibody is at a concentration of about 100 mg/ml;
histidine;
sucrose; and
a polysorbate.

20. The composition according to claim 19, wherein
histidine is present at a concentration of from about 5 mM to about 50 mM;
sucrose is present at a concentration of from about 50 mg/ml to about 100 mg/ml; and
the polysorbate is polysorbate 80 and is present in a concentration of from about 0.1 to about 10 mg/ml.
